(19) United States
(12) Patent Application Publication
(10) Pub. No.: US 2009/0143455 A1
(43) Pub. Date: Jun. 4, 2009

(54) Pyrazoles Useful in the Treatment of Inflammation

(76) Inventors: Benjamin Pelcman, Solna (SE); Andrei Sanin, Solna (SE); Peter Nilsson, Solna (SE); Thomas Boesen, Copenhagen (DK); Stine Byskov Vogensen, Copenhagen (DK); Hasse Kromann, Copenhagen (DK); Thomas Groth, Copenhagen (DK)

Correspondence Address: MORGAN LEWIS & BOCKIUS LLP, 1111 PENNSYLVANIA AVENUE NW, WASHINGTON, DC, 20004, US

(21) Appl. No.: 12/083,782

(22) PCT Filed: Oct. 19, 2006
(86) PCT No.: PCT/GB2006/003876

§ 371 Date: Oct. 1, 2008

Related U.S. Application Data
(60) Provisional application No. 60/728,300, filed on Oct. 20, 2005.

(51) Int. Cl. A61K 31/415; C07D 231/10; A61P 29/00; A61P 11/00; A61P 1/00; A61P 9/00; A61P 17/00; A61P 19/02; A61P 25/00; A61P 35/00

(52) U.S. Cl. 514/406; 548/374.1

(57) ABSTRACT

There is provided compounds of formula I,

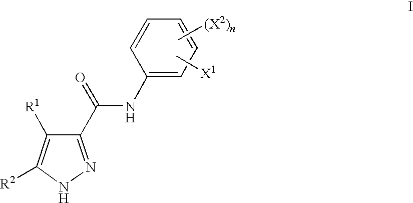

wherein R¹, R², X¹, X² and n have meanings given in the description, and pharmaceutically-acceptable salts thereof, which compounds are useful in the treatment of diseases in which inhibition of the activity of a lipoxygenase (e.g. 15-lipoxygenase) is desired and/or required, and particularly in the treatment of inflammation.

### FIELD OF THE INVENTION

This invention relates to compounds for use as pharmaceuticals, some of which compounds are novel and some of which are known. The invention further relates to the use of such compounds in the inhibition of the activity of lipoxygenases, such as 15-lipoxygenase, and thus in the treatment of inflammatory diseases and of inflammation generally. The invention also relates to newt compounds that are useful in that inhibition, to pharmaceutical compositions containing such compounds, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, a new and/or alternative anti-inflammatory treatment would be of benefit to all of the above-mentioned patient groups. In particular, there is a real and substantial unmet clinical need for an effective anti-inflammatory drug capable of treating inflammatory disorders, such as asthma, with no real or perceived side effects.

The mammalian lipoxygenases are a family of structurally-related enzymes, which catalyze the oxygenation of inter alia arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LThRas).

Another class of enzymes that metabolize arachidonic acid are the cyclooxygenases. Arachidonic acid metabolites that are produced by this process include prostagialins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflammation.

### PRIOR ART

Certain pyrazole compounds that are structurally related to those described herein are commercially available. However, to the knowledge of the applicant, these compounds have never been disclosed in any printed publication and as such have no perceived utility ascribed to them.

JP 2-129171 discloses various N-unsubstituted 5-trifluoromethylpyrazole-based agrochemicals. The use of these compounds as pharmaceuticals is neither mentioned nor suggested.

Pyrazole-based compounds have been disclosed in several publications. For example, international patent application WO 01/57024 discloses various pyrazoles that are useful in blockading voltage-dependent sodium channels; international applications WO 03/020217 and WO 01/58869, and U.S. Patent No. 2004/0192667 disclose various nitrogen-containing heterocycles, including pyrazoles, that are useful as modulators of cannabinoid receptors; international patent application WO 99/20294 discloses pyrazoles that are useful in the treatment of cystic fibrosis; international application WO 2005/007625 discloses anti-tuberculosis compounds that include pyrazoles; U.S. patent no. 2003/0091116 and international patent applications WO 01/19798, WO 99/32454 and WO 2004/055815 disclose inter alia pyrazoles that may be useful as Factor Xa inhibitors; and WO 01/21160 discloses antiviral compounds that include pyrazoles. There is no disclosure in any of these documents of 1(N)-unsubstituted-3-amidopyrazoles for use in treating inflammation and/or as inhibitors of lipoxygenases.

International patent application WO 97/19062 discloses various pyrazoles for the treatment of skin related diseases and further mentions the use of such compounds in the treatment of various inflammatory diseases. However, this document does not mention or suggest 3-amido pyrazoles that are substituted at the 4- and/or 5-position of the pyrazole ring with a halo or trifluoromethyl group.

International patent application WO 2004/096795 discloses various heterocycles, including pyrazoles, as inhibitors of protein tyrosine kinases and international patent application WO 01/55115 discloses various aromatic amides that may be useful as activators of caspases and inducers of apoptosis. Accordingly, the compounds disclosed in these documents may be useful in the treatment of inter alia cancer. There is no disclosure or suggestion in either of these documents of the use of such compounds as inhibitors of lipoxygenases.

International patent application WO 2005/016877 discloses pyrazoles that may be useful in the inhibition of 11β-hydroxysteroid dehydrogenase-1 (and therefore useful in the treatment of inter alia diabetes). There is no specific disclosure in this document of pyrazoles that are substituted in the 3-position with an aromatic amido group.

Certain pyrazolecarboxylic acid hydrazides, structurally unrelated to the compounds described herein, have been disclosed as anti-inflammatory agents in Tihanyi et al, Eur. J. Med. Chem.—Chim. Yher., 1984, 19, 433 and Goel et al, J. Chem. Inf. Comput. Sci. 1995, 35, 510.

Vertuani et al., Journal of Pharmaceutical Sciences, Vol. 74, No. 9 (1985) discloses various pyrazoles that possess anti-inflammatory and analgesic activities. There is no mention or suggestion of pyrazoles that are substituted on the pyrazole ring itself with a chloro, fluoro or trifluoromethyl group.

International patent application WO 03/037274 discloses various pyrazoles that may be useful in treating inflammatory pain, which mechanism works by blocking sodium channels. This document relates primarily to pyrazoles that are 1(N)-substituted and also to pyrazoles that are substituted by an amido group in the 4-position.

International patent application WO 03/068767 also relates to inter alia pyrazole-containing compounds that may be useful in treating inflammatory pain by opening potassium ion channels. However, this document relates specifically to pyrimidinyl amido compounds.

International patent applications WO 2004/080999 and WO 2006/032852 both disclose various 3-amidopyrazoles for use in the treatment of inflammation. However, there is no disclosure or suggestion in any of these documents of N-unsubstituted 3-amidopyrazoles for use in such treatment.

International patent application WO 2006/032851 discloses various 3-amidopyrazoles for use in the treatment of inflammation in which the amido group is substituted with a bicyclic heterocyclic group. However, there is no disclosure or suggestion of corresponding 3-amidopyrazoles in which the amido group is substituted by a monocyclic aromatic group.

### DISCLOSURE OF THE INVENTION

According to the invention there is provided a compound of formula I,

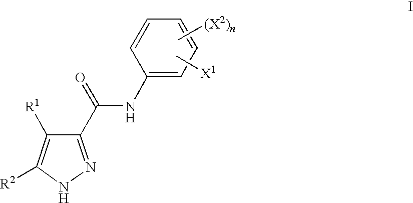

wherein,
R¹ and R² independently represent H, Cl, F, CHF₂ or CF₃, provided that at least one of R¹ and R² does not represent H;
X¹ represents halo, —R^{3a}, —OR^{3q} or —S(O)₂N(R^{4j})R^{5j};
X² represents halo, —R^{3a}, —CN, —C(O)R^{3b}, —C(O)OR^{3c}, —C(O)N(R^{4a})R^{5a}, —N(R^{4b})R^{5b}, N(R^{3d})C(O)R^{4c}, N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —S(O)₂N(R^{4h})R^{5h}, —S(O)₂OH, —N(R^{3k})S(O)₂R^{3m}, —OC(O)R³ⁿ, —OC(O)OR^{3p} or —P(O)(OR⁴ⁱ)(OR⁵ⁱ);
n represents 0, 1, 2, 3 or 4;
m represents 0, 1 or 2;
R^{3a} represents, on each occasion when used herein, C₁₋₆ alkyl optionally substituted by one or more substituents selected from F, Cl, —N(R^{4b})R^{5b}, —N₃, ═O and —OR^{3h};
R^{3b} to R^{3h} (in the case of R^{3h} on each occasion when used herein), R^{3k}, R³ⁿ, R^{3q}, R^{4a} to R^{4j} (in the case of R^{4b} on each occasion where used herein), R^{5a}, R^{5b} (on each occasion when used herein), R^{5d} and R^{5f} to R^{5j} independently represent hydrogen or C₁₋₆ alkyl optionally substituted by one or more substituents selected from F, Cl, —OCH₃, —OCH₂CH₃, —OCHF₂, and —OCF₃; or
any of the pairs R^{4a} and R^{5a}, R^{4b} and R^{5b}, R^{4d} and R^{5d}, R^{4f} and R^{5f}, R^{4g} and R^{5g}, R^{4h} and R^{5h}, and R^{4j} and R^{5j}, may be linked together to form a 3- to 6-membered ring, which ring optionally contains a fiber heteroatom (such as nitrogen or oxygen) in addition to the nitrogen atom to which these substituents are necessarily attached, and which ring is optionally substituted by ═O and/or C₁₋₆ alkyl, which alkyl group is optionally substituted by one or more F atom;
R³ⁱ, R^{3j}, R^{3m} and R^{3p} independently represent C₁₋₆ alkyl optionally substituted by one or more substituents selected from F, Cl, —OCH₃, —OCH₂CH₃, —OCHF₂, and —OCF₃,
or a pharmaceutically-acceptable salt thereof, for use as a pharmaceutical.

Compounds of formula I, or pharmaceutically-acceptable salts thereof, may be in isolated (i.e. ex vivo) form.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Compounds of formula I may contain double bonds and may thus exist as E (entgegen) and Z (zusammen) geometric isomers about each individual double bond. All such isomers and mixtures thereof are included within the scope of the invention.

Compounds of formula I may also exhibit tautomerism. All tautomeric forms and mixtures thereof are included within the scope of the invention.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming, in the case of alkyl, a C_{3-q} cycloalkyl group). Further, when there is a sufficient number (i.e. a minimum of four) of carbon atoms, such groups may also be part cyclic/acyclic. Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alkynyl group).

The term “halo”, when used herein, includes fluoro, chloro, bromo and iodo.

In compounds of formula I, the skilled person will appreciate that —(X²)ₙ represents one to four optional (given that n may represent 0) substituents. In the case, where n represents 2, 3 or 4, i.e. when there are 2, 3 or 4 separate X² substituents present these substituents are in no way interdependent, i.e. in the case when n represents 2, the two X² substituents may represent the same or different groups.

For the avoidance of doubt, when a phrase such as “R^{3b} to R^{3h}” is employed herein, this will be understood by the skilled person to mean R^{3b}, R^{3c}, R^{3d}, R^{3e}, R^{3f}, R^{3g} and R^{3h} inclusively.

For the avoidance of doubt, in cases in which the identity of two or more substituents in a compound of formula I may be the same, the actual identities of the respective substituents are not in any way interdependent. For example, in the situation in which X¹ and X² are both R^{3a}, in which R^{1a} is a C₁₋₆ alkyl group, the respective alkyl groups may be the same or different. Similarly, when groups are substituted by more than one substituent as defined herein, the identities of those individual substituents are not to be regarded as being interdependent. For example, when X¹ represents C₁₋₆ alkyl substituted by —OR^{3h} and X² represents —OR^{3h}, then the identity of the two —OR^{3h} groups are not to be regarded as being interdependent.

Compounds of the invention that may be mentioned include those in which:

X¹ represents —OR^{3q} or, preferably, halo or —R^{3a}; and/or
R¹ and R² independently represent H, Cl, F or CF₃.

Further compounds of the invention that may be mentioned include those in which:

R^{4b} and R^{5b} are not linked together as hereinbefore defined;
when n represents 1, 2, 3 or 4 and at least one of the X² substituents is located at the ortho position (relative to the point of attachment of the phenyl ring to the —N(H)C(O)— group of the compound of formula I), then X² represents halo, —R³¹, —CN, —C(O)R^{3b}, —C(O)OR^{3c}, —C(O)N(R^{4a})R^{5a}, —N₃, —NO₂, —OR^{3b}, —OC(O)N(R^{4g})R^{5g}, —OS(O)₂R³ⁱ, S(O)ₘR^{3j}, —S(O)₂N(R^{4h})R^{5h}, —S(O)₂OH, —OC(O)R³ⁿ, —OC(O)OR^{3p} or —P(O)(OR⁴ⁱ)(OR⁵ⁱ);
when X¹ represents an ortho substituent and/or (when n is 1, 2, 3 or 4) there is a X² substituent located at an ortho position (relative to the point of attachment of the phenyl ring to the —N(H)C(O)— group of the compound of formula I), then X¹ represents —S(O)₂N(R^{4j})R^{5j} or, preferably, halo and/or X² represents halo, —CN, C(O)R^{3b}, —C(O)OR^{3c}, —C(O)N(R^{4a})R^{5a}, —N(R^{4a})R^{5b}, N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OC(O)N(R^{4g})R^{5g}, —OS(O)₂R³ⁱ, —S(O)₂N(R^{4h})R^{5h}, —S(O)₂OH, —N(R^{3k})S(O)₂R^{3m}, —OC(O)R³ⁿ, —OC(O)OR^{3p} or —P(O)(OR⁴ⁱ)(OR⁵ⁱ).

Preferred compounds of formula I include those in which R¹ and R² independently represent H, F or Cl.

More preferred compounds of formula I include those in which:

n is 2 or 3 (e.g. 2) or, more preferably, 0 or 1 (e.g. 1);
when any of the pairs R^{4a} and R^{5a}, R^{4b} and R^{5b}, R^{4d} and R^{5d}, R^{4f} and R^{5f}, R^{4g} and R^{5g}, R^{4h} and R^{5h}, and R^{4j} and R^{5j}, are linked together, they form a 5- to 6-membered ring, which ring optionally contains a further heteroatom (such as nitrogen or oxygen) and is optionally substituted by methyl, —CHF₂ or CF₃ (so forming, for example, a pyrrolidinyl, piperidinyl, morpholinyl or a piperazinyl (e.g. 4-methylpiperazinyl) ring);
R^{3a} represents C₁₋₆ alkyl optionally substituted by one or more substituents selected from F and —OR^{3h}.

Further preferred compounds of formula I include those in which:

R¹ represents CF₃ or, more preferably, H, Cl or F;
R² represents CHF₂ or, more preferably, H, Cl or CF₃;
when R¹ represents Cl, then R² represents CHF₂, CF₃ or, more preferably, H or Cl;
when R¹ represents H, then R² represents Cl or CF₃;
when R¹ represents F, then R² represents H;
when R¹ represents CF₃, then R² represents H or CF₃;
when R² represents H, then R¹ represents CF₃ or, more preferably, Cl or F;
when R² represents Cl, then R¹ represents H or Cl;
when R² represents CF₃, then R¹ represents Cl, CF₃ or, more preferably, H;
when R² represents CHF₂, then R¹ represents CL;
X¹ represents —S(O)₂N(R^{4j})R^{5j}, preferably, —OR^{3q} or, more preferably, F, Cl or R^{3a} (such as C₁₋₃ (e.g. C₁₋₂) alkyl (e.g. methyl), optionally substituted by one or more fluoro atoms (so forming, for example, a —CHF₂ or CF₃ group));
X² represents F, Cl, Br, —R^{3a}, —CN, —C(O)R^{3b}, —C(O)OR^{3c}, —C(O)N(R^{4a})R^{5a}, N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j} or —S(O)₂N(R^{4h})R^{5h};
R^{3a} represents C₁₋₄ alkyl (e.g. ethyl, isopropyl, t-butyl, cyclopropyl, cyclobutyl, cyclopropylmethyl or, especially, methyl) optionally substituted by one or more F atoms (so forming, for example, a —CHF₂ or CF₃ group);

R^{3b}, R^{3c}, R^{3h}, R^{4a} to R^{4h}, R^{4j}, R^{5a}, R^{5b}, R^{5d}, R^{5f} to R^{5h} and R^{5j} independently represent hydrogen or C₁₋₄ alkyl (e.g. methyl), or the relevant pairs (i.e. R^{4j} and R^{5j}, and preferably, R^{4a} and R^{5a}, R^{4b} and R^{5b}, R^{4d} and R^{5d}, R^{4f} and R^{5f}, R^{4g} and R^{4g} and R^{4h} and R^{5h}) may be linked together as hereinbefore defined;

R^{3d} to R^{3g} independently represent C₁₋₂ alkyl (e.g. methyl) or, more particularly, hydrogen;
R³ⁱ and R^{3j} independently represent C₁₋₄ (e.g. C₁₋₂) alkyl (e.g. methyl) optionally substituted by one or more F atoms (so forming, for example a CF₃ group)
R^{3q} represents C₁₋₄ (e.g. C₁₋₂) alkyl (e.g. methyl), which alkyl group is unsubstituted or, more preferably, substituted by one or more fluoro atoms (so forming, for example, a —CHF₂ or —CF₃ group).

More preferred compounds of formula I includes those in which:

X¹ represents —OCF₃, —OCHF₂, —S(O)₂N)CH₃, —S(O)₂N(CH₃)₂ or, more preferably, F, Cl, CH₃ or CF₃;
X² represents —CN, —C(O)N(R^{4a})R^{5a}, —N(R^{4b})R^{5b}, —N(H)C(O)R^{4c}, —S(O)₂CH₃, —S(O)₂CF₃, —S(O)₂N(R^{4h})R^{5h} or, more preferably F, Cl, R^{3a} or —OR^{3h};
R^{3a} represents isopropyl (which group is preferably unsubstituted) or methyl (which group is optionally substituted as hereinbefore defined);
R^{3h} represents hydrogen or C₁₋₄ alkyl (e.g. ethyl, isopropyl, t-butyl, cyclopropyl, cyclobutyl, cyclopropylmethyl or, more preferably, methyl) optionally substituted by one or more fluoro atoms (so forming, for example, —CHF₂ or CF₃);
R^{4a}, R^{4b}, R^{4c}, R^{4h}, R^{5a}, R^{5b} and R^{5h} independently represent hydrogen, methyl or ethyl, or the relevant pairs (i.e. R^{4a} and R^{5a}, R^{4b} and R^{5b} and R^{4b} and R^{5h}) are linked together to form a pyrrolidinyl, piperidinyl morpholinyl or a 4-methylpiperazinyl ring;
R^{4j} and R^{5j} may be linked together as hereinbefore described (e.g. to form a pyrrolidinyl, piperidinyl, morpholinyl or a 4-methylpiperazinyl ring) or may independently represent ethyl or, more preferably, hydrogen or methyl.

Further preferred compounds of formula I include those in which X¹ is selected from —OCF₃, —OCHF₂, —S(O)₂N(H)CH₃, —S(O)₂N(CH₃)₂ and, more preferably, F, Cl and CF₃, and (X²), is either not present (i.e. n represents 0) or, more preferably, represents a single substituent selected from isopropyl or, more particularly, F, Cl, CF₃, methyl and methoxy.

Yet more preferred compounds of formula I include those in which:

X¹ is in the 2- or, more preferably, 3- or, particularly, 4-position relative to the point of attachment of the phenyl ring to the rest of the compound of formula I and/or preferably represents —OCF₃, —OCHF₂, —S(O)₂N(H)CH₃, —S(O)₂N(CH₃)₂ or, more preferably, F or Cl;
X² is either not present or, more particularly, represents isopropyl or, preferably, F or Cl and/or is in the 4- or, preferably, the 2-position

Particularly preferred compounds of formula I include those of the examples described hereinafter.

Compounds of formula I may be made in accordance with techniques that are well known to those skilled in the art, for example as described hereinafter.

According to a further aspect of the invention there is provided a process for the preparation of a compound of formula I, which process comprises:

(i) For compounds of formula I in which R¹ represents CHF₂, Cl, F or CF₃, reaction of a corresponding compound of formula I in which R² represents hydrogen, with an appropriate base (or a mixture of bases), such as potassium bis(trimethylsilyl)amide, sodium bis(trimethylsilyl)amide, sodium hydride, potassium tert-butoxide or an organolithium base, such as n-BuLi, s-BuLi t-BuLi, lithium diisopropylamide or lithium 2,2,6,6-tetramethylpiperidine (which organolithium base is optionally in the presence of an additive (for example, a lithium coordinating agent such as an ether (e.g. dimethoxyethane) or an amine (e.g. tetramethylethylenediamine (WANDA), (−)sparteine or 1,3-dimethyl-3,4,5,6-tetrahydro-2(1H)-pyrimidinone (DMPU) and the like)) followed by quenching with an appropriate electrophile such as:
(a) for compounds of formula I in which R² represents CHF₂ or CF₃, a compound of formula II,

R^{c}L^{1a} II
wherein R^{c} represents CHF₂ or CF₃, L^{1a} represents a suitable leaving group such as halo (e.g. iodo or bromo) or a sulfonate group (such as —OSO₂CF₃, OSO₂CH₃ and —OSO₂-aryl (e.g. —O-tosyl)). When the compound of formula II is a trifluoromethylating agent, it may be a dibenzothiophenium tetrafluoroborate (e.g. 5-(trifluoromethyl)-dibenzothiophenium tetrafluoroborate);
(b) for compounds of formula I in which R² represents Cl or F, an electrophile that provides a source of these atoms. For example, for chlorine atoms reagents include N-chlorosuccinimide, chlorine, iodine monochloride and hexachloroethane and for fluorine atoms reagents include xenon difluoride, SELECTFLUOR® ([1-(chloromethyl)-4-fluoro-1,4-diazoniabicyclo[2.2.2]octane bis-(tetrafluoroborate)]), CF₃OF, perchloryl fluoride, F₂ and acetylhypofluoride.

The skilled person will appreciate that the corresponding compounds of formula I in which R² represents hydrogen (on which the above reaction is performed) may need to be protected at the nitrogen atom of the pyrazole ring system, preferably with a protective group that is also a directing metallation group (such as a benzenesulfonyl group or a SEM (i.e. a —CH₂OC₂H₄Si(CH₃)₃) group. The reaction may be performed in the presence of a suitable solvent, such as a polar aprotic solvent (e.g. tetrahydrofuran or diethyl ether), at sub-ambient temperatures (e.g. 0° C. to −78° C.) under an inert atmosphere followed (as appropriate) by deprotection of the N-protective group under standard conditions (e.g. when a benzenesulfonyl group is employed, by hydrolysis or, when a SEM group is employed by reaction in the presence of HCl in EtOH).

(ii) For compounds of formula I in which R² represents CF₃, reaction of a compound corresponding to a compound of formula I but in which R² represents bromo or, preferably, iodo with CuCF₃ (or a source of CuCF₃) in, for example, the presence of HMPA and DMF. The skilled person will appreciate that the reagent CuCF₃ may not be isolated as such, and may be prepared in accordance with the procedures described in Burton D. G.; Wiemers D. M.; J. Am. Chem. Soc., 1985, 107, 5014-5015 and Mawson S. D.; Weavers R. T.; Tetrahedron Letters., 1993, Vol. 34, No. 19, 3139-3140 (for example, by the reaction of zinc and e.g. CF₂Br₂ in DMF so forming ZnCF₃ (or a source thereof) followed by treatment with CuBr in HMPA).
(iii) Reaction of a compound of formula III,

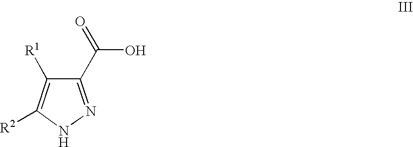

or a N-protected and/or O-protected (e.g. ester) derivative thereof, wherein R¹ and R² are as hereinbefore defined, with a compound of formula IV,

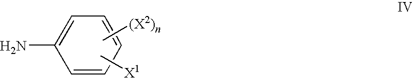

wherein X¹, X² and n are as hereinbefore defined under coupling conditions, for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, triethylamine, dimethylaminopyridine, diisopropylamine, diisopropylethyl-amine, 1,8-diazabicyclo[5.4.0]undec-7-ene, sodium hydroxide, N-ethyldiisopropylamine, N-(methylpolystyrene)-4-(methylamino)pyridine, butyllithium (e.g. n-, s- or t-butyllithium) or mixtures thereof), an appropriate solvent (e.g. tetrahydrofuran, pyridine, toluene, dichloromethane, chloroform, acetonitrile, dimethylformamide, dimethylsulfoxide, water or triethylamine) and a suitable coupling agent (e.g. 1,1′-carbonyldiimidazole, N,N-dicyclohexylcarbodiimide, 1-(3-dimethylamino-propyl)-3-ethylcarbodiimide (or hydrochloride thereof), N,N-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate, benzotriazol-1-yloxytrispyrrolidinophosphonium hexafluorophosphate, bromo-tris-pyrrolidinophosphonium hexafluorophosphate, 2-(1H-benzotriazol-1-yl)-1,1,3,3-tetramethyluronium tetrafluorocarbonate, 1-cyclohexylcarbodiimide-3-propyloxymethyl polystyrene, O-(7-azabenzotriazol-1-yl)-N,N,N,N′-tetramethyluronium hexafluorophosphate or O-benzotriazol-1-yl-N,N,N′,N′-tetramethyluronium tetrafluoroborate). Alternatively, compounds of formula III may first be activated by treatment with a suitable reagent (e.g. oxalyl chloride, thionyl chloride, etc) optionally in the presence of an appropriate solvent (e.g. dichloromethane, THF, toluene or benzene) and a suitable catalyst (e.g. DMF), resulting in the formation of the respective acyl chloride. This activated intermediate may then be reacted with a compound of formula IV under standard conditions, such as those described above. The skilled person will appreciate that when compounds of formula IV are liquid in nature, they may serve as both solvent and reactant in this reaction. Alternative methods of performing this step include reaction of an O-protected derivative (e.g. an ethyl ester) of a compound of formula III with a compound of formula IV, which latter compound may first be treated with an appropriate reagent (e.g. trimethylaluminium), for example in an inert atmosphere and in the presence of a suitable solvent (e.g. dichloromethane).
(iv) Reaction of a compound of formula V,

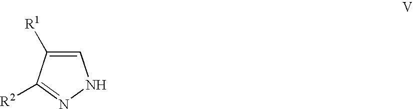

wherein R¹ and R² are as hereinbefore defined, with a suitable base, such as one described in process step (i) above, followed by reaction with a compound of formula VI,

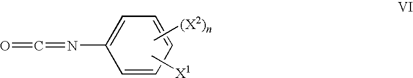

wherein X¹, X² and n are as hereinbefore defined, followed by quenching with a suitable proton source (e.g. water or sat, aq. NH₄Cl solution). This reaction may be performed under similar conditions to those described above in respect of process step (i). The skilled person will therefore appreciate that the pyrazole nitrogen may need to be protected.
(v) For compounds of formula I in which R² represents hydrogen and R¹ is as hereinbefore defined, removal of the group J from a compound of formula VII,

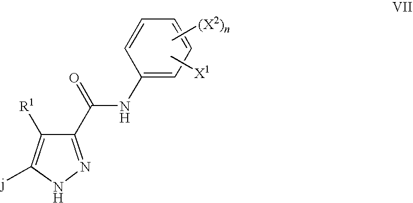

wherein J represents —Si(R^{t})₃ or —Sn(R^{z})₃ (in which each R^{t} independently represents a C₁₋₆ alkyl (e.g. a methyl or isopropyl) group or an aryl (e.g. phenyl) group and each R^{z} independently represents C₁₋₆ alkyl (e.g. methyl or butyl)), and R¹, X¹, X² and n are as hereinbefore defined. When J represents —Si(t)₃, the reaction may be performed in the presence of an appropriate reagent for the removal of the silyl group, such as a source of halide anions (e.g. tetrabutylammonium fluoride, tetramethylammonium fluoride, hydrogen fluoride or potassium fluoride), for example, in the presence of a suitable solvent (e.g. tetrahydrofuran) at room temperature. When J represents —Sn(R^{z})₃, the reaction may be a standard hydrolysis, for example reaction with water or an aqueous acid (e.g. hydrochloric acid) in the presence of an appropriate solvent (e.g. dioxane, tetrahydrofuran, MeOH or EtOH (or mixtures thererof)).
(vi) Reaction of a compound of formula VIII,

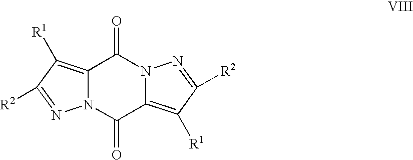

wherein R¹ and R² are as hereinbefore defined, with a compound of formula IV as hereinbefore defined, for example under coupling conditions such as those described hereinbefore in respect of process step (iii) above. Preferred conditions include reaction in the presence of base, solvent but no coupling reagent. In this case, the compound of formula IV may also be employed in excess.
(vii) For compounds of formula I in which one of R¹ or R² represents CHF₂, CF₃, Cl or F and the other represents H, reaction of a compound corresponding to a compound of formula I but in which one of R¹ or R² represents bromo or iodo and the other represents H (as appropriate) with a suitable organolithium base (e.g. t-BuLi, s-BuLi or n-BuLi) optionally in the presence of an additive (such as one hereinbefore described in respect of process step (i)), followed by quenching with a compound of formula II, as hereinbefore defined, or a source of chlorine or fluorine atoms, such as one described in respect of process (i) above. This reaction may be performed in the presence of a suitable solvent, such as one hereinbefore described in respect of process step (i) at low temperatures (e.g. −78 to −120° C.) under an inert atmosphere.
(viii) Reaction of a compound of formula VIIIA

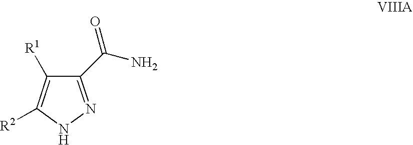

or a N-protected (e.g. at the pyrazole nitrogen) derivative thereof, wherein R¹ and R² are as hereinbefore defined, with a compound of formula VIIIB,

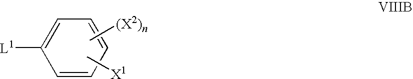

wherein L¹ represents a suitable leaving group, such as halo (e.g. chloro, bromo and iodo), —OSO₂CF₃, —B(OH)₂, —Sn(R^{z})₃ (wherein R^{z} is as hereinbefore defined), —Pb(OC(O)CH₃)₃, —Bi(W)₂, —Bi(W)₂(OC(O)CH₃)₂, —Bi(W)₂(OC(O)CF₃)₂ or —I(W)(BF₄), and W represents an aryl or heteroaryl group, both of which are optionally substituted by one or more groups selected from X² as hereinbefore defined (e.g. W represents the phenyl ring of the compound of formula I as hereinbefore defined), and X¹, X² and n are as hereinbefore defined, for example in the presence of a catalyst containing, preferably, Pd or Cu, and a base, such as potassium or sodium hydroxide, potassium carbonate, potassium tert-butoxide and lithium N,N-diisopropylamide. Catalysts that may be mentioned include Pd₂(dba)₃ (tris(dibenzylideneacetone)dipalladium(0)), bases that may be mentioned include cesium carbonate, ligands that may be mentioned include 2,2′-bis(diphenylphosphino)-1,1′-binaphthyl and solvents that may be employed include toluene. Such reactions may be performed at elevated temperature (e.g. at about 90° C.) under an inert (e.g. argon) atmosphere.

Compounds of formula III (or derivatives thereof) in which R² represents H or CF₃ may be prepared by reaction of a compound of formula IX,

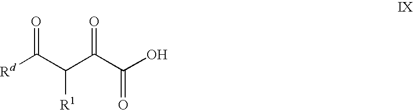

or an enol ether equivalent (e.g. a methyl enol ether or a silyl (e.g. trimethylsilyl) enol ether), or an O-protected (e.g. at the carboxylic acid) derivative thereof, wherein R^{d} represents H or CF₃ and R¹ is as hereinbefore defined, with hydrazine (or a hydrate or derivative (e.g. benzylhydrazine) thereof), for example in the presence of an alcoholic solvent (e.g. EtOH) at elevated temperature (e.g. at reflux).

Compounds of formula III in which either one of R¹ or R² represents Cl or F and the other represents CHF₂, H or CF₃ or both R¹ and R² represent Cl or F, may be prepared by reaction of a corresponding compound of formula III in which R¹ and R² both represent H or one of R¹ or R² represents H and the other represents CHF₂ or CF₃, with an electrophile that provides a source of chlorine or fluorine atoms, such as one described hereinbefore in respect of preparation of compounds of formula I (process step (i)(b) above), under reaction conditions known to those skilled in the art such as in the presence of a suitable solvent (e.g. water). Thus, relevant 4-halo, 5-halo or 4,5-dihalo substituted pyrazoles may be prepared in such a manner.

Compounds of formula III in which one of R¹ or R² represents fluoro and the other represents H may be prepared from 4-nitropyrazole-3-carboxylic acid or 5-nitropyrazole-3-carboxylic acid (as appropriate) employing an appropriate reagent for the conversion of the nitro group to a fluoro group (such as sodium fluoride, potassium fluoride, tetramethylammonium fluoride or tetrabutylammonium fluoride) under conditions known to those skilled in the art.

Compounds of formula III in which one of R¹ or R² represents Cl or F and the other represents H, may be prepared by reaction of a compound corresponding to a compound of formula III but in which one of R¹ or R² represents amino and the other represents H (as appropriate; i.e. 4- or 5-aminopyrazole-3-carboxylic acid) followed by conversion of the amino group to a diazonium salt (employing reagents and conditions known to those skilled in the art, e.g. NaNO₂ and HCl at 5° C.) and then the addition of an appropriate nucleophile for the conversion to a Cl or F. Suitable nucleophiles include potassium, sodium or copper chlorides or fluorides. Alternatively, for the introduction of the fluoro group, the appropriate diazonium salt may be treated with a compound that provides a source of fluoroborate (e.g. tetrafluoroborate) salts, for example by introducing a cold aqueous solution of NaBF₄, HBF₄ or NH₄BF₄, so forming the appropriate diazonium fluoroborate (e.g. diazonium tetraflouorborate), which may then be heated

Compounds of formula III in which R¹ represents CHF₂, F, Cl or CF₃ may be prepared from corresponding compounds of formula III in which R¹ represents H, for example in accordance with a procedure described in R. Storer et al., Nucleosides & Nucleotides 18, 203 (1999). The appropriate reagents that may be employed for the introduction of the CHF₂, F, Cl or CF₃ group are described hereinbefore in respect of preparation of compounds of formula I (process step (i) above).

Compounds of formula III may alternatively be prepared by oxidation of a compound of formula X,

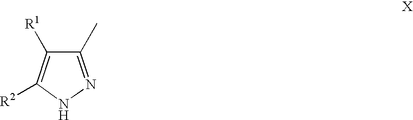

wherein R¹ and R² are as hereinbefore defined, under oxidation conditions known to those skilled in the art, for example mild or strong (e.g. employing an aqueous solution of potassium permanganate and heating at reflux) oxidation conditions as appropriate.

Compounds of formula III in which R² is as hereinbefore defined (e.g. H, Cl or F) may be prepared by reaction of a compound of formula XI,

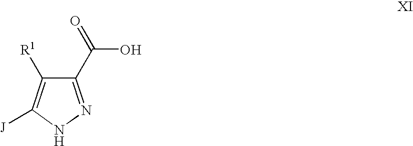

or a N-protected and/or O-protected (e.g. ester) derivative thereof, wherein J and R¹ are as hereinbefore defined. For compounds of formula III in which R² represents Cl or F, reaction may be with a suitable halogenating (i.e. chlorinating or fluorinating) reagent such as cesium fluoroxysulfate or one described hereinbefore in respect of process step (i)(b), optionally in the presence of a suitable solvent (e.g. hexane, diethyl ether, tetrahydrofuran or 1,4-dioxane or mixtures thereof) under conditions known to those skilled in the art. For compounds of formula III in which R² represents H, reaction may be with reagents and under conditions such as those hereinbefore described in respect of preparation of compounds of formula I (process step (v)).

Compounds of formula III, in which R¹ and R² are as hereinbefore defined, may be prepared by oxidation of a compound of formula XIA,

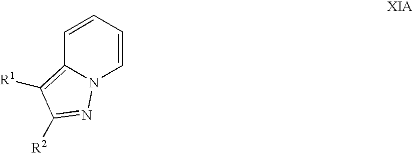

wherein R¹ and R² are as hereinbefore defined, under oxidation conditions known to those skilled in the art, such as those described hereinbefore in respect of preparation of compounds of formula III (i.e. from a compound of formula X) above.

Compounds of formula III (or protected derivatives thereof) in which R² represents H and R¹ is as hereinbefore defined (and preferably represents Cl or F) and may be prepared by reaction of a compound of formula XIB,

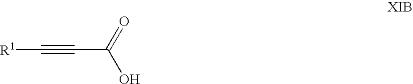

or a protected derivative (e.g. an ester, such as a C₁₋₆ (e.g. ethyl) ester) thereof, wherein R¹ is as hereinbefore defined (and preferably represents Cl or F), with diazomethane, or a protected derivative thereof (e.g. trimethylsilyldiazomethane), for example under conditions known to those skilled in the art (such as in the presence of a suitable solvent (e.g. diethyl ether) and/or at low temperatures (e.g. 0° C. to room temperature)).

Compounds of formula III or X may be prepared by reaction of a corresponding compound of formula V with a suitable base, such as one described in respect of preparation of compounds of formula I, process step (i) (and, in particular, organolithiums) followed by reaction with an appropriate electrophile. For example, in the case of compounds of formula III, for the introduction of a carboxylic acid group (or a protected derivative thereof), the electrophile may be a source of CO₂ (e.g. CO₂ gas), which addition is followed by the addition of a suitable proton source (e.g. HCl), or a compound of formula XV as defined hereinafter (e.g. methyl or ethyl chloroformate) or, in the case of compounds of formula X, a compound of formula XVI as defined hereinafter (e.g. methyl iodide), or the like.

Compounds of formula IV in which X¹ represents —SO₂N(R^{4j})R^{5j} and X², n, R^{4j} and R^{5j} are as hereinbefore defined, may be prepared by reaction of a compound of formula XIC,

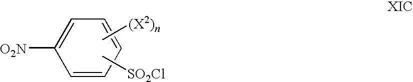

wherein X² and n are as hereinbefore defined, with a compound of formula XID,

H₂N(R^{4j})R^{5j} XID

wherein R^{4j} and R^{5j} are as hereinbefore defined, for example under conditions known to those skilled in the art (such as in the presence of a suitable base (e.g. triethylamine) and a suitable solvent (e.g. dichloromethane)), followed by hydrogenation of the isolated nitro intermediate, for example under conditions known to those skilled in the art (such as in the presence of a suitable catalyst (e.g. Pd on carbon (10%)) and a suitable solvent (e.g. MeOH)).

Compounds of formula V may be prepared from a compound of formula XIE,

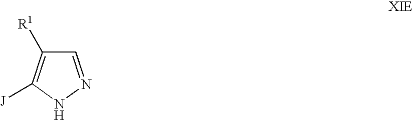

or a N-protected derivative thereof, wherein J and R¹ are as hereinbefore defined, using reagents and procedures known to those skilled in the at for example such as those hereinbefore described in respect of preparation of compounds of formula I (process route (v)), or in respect of preparation of compounds of formula III (the process involving reaction with a compound of formula XI).

Compounds of formula VII may be prepared by reaction of a compound of formula IV as hereinbefore defined with either:

(I) a compound of formula XII,

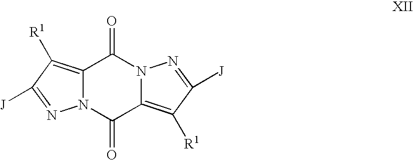

wherein R¹ and J are as hereinbefore defined; or
(II) a compound of formula XI as hereinbefore defined (or a N-protected and/or O-protected (e.g. ester) derivative thereof), for example under coupling conditions similar to those described hereinbefore in respect of preparation of compounds of formula I (process step (iii) or (vi) above).

Compounds of formulae VIII and XII may be prepared from compounds of formula III, and compounds of formula XI, respectively, under dimerising conditions, for example in the presence of thionyl chloride or oxalyl chloride (optionally in the presence of a suitable solvent and catalyst, such as one hereinbefore defined in respect of process step (iii)). Other dimerising reagents include carbodiimides, such as 1,3-dicyclohexylcarbodiimide or 1-(3-dimethylaminopropyl)-3-ethylcarbodiimide (EDCI, or hydrochloride thereof) optionally in the presence of a suitable base (e.g. 4-dimethylaminopyridine).

Compounds of formula X in which R² represents CHF₂, Cl, F or CF₃ may be prepared from a corresponding compound of formula X (or a protected derivative thereof) in which R¹ represent H, for example under conditions and employing reagents such as those described hereinbefore in respect of preparation of compounds of formula I (process step (i) above).

Alternatively, compounds of formula X may be prepared by N-dealkylation of a compound of formula XIIA,

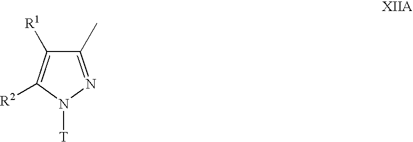

wherein T represents optionally substituted C₁₋₆ alkyl (e.g. methyl) and R¹ and R² are as hereinbefore defined, under dealkylation conditions known to those skilled in the art, for example by reaction with a suitable reagent (e.g. pyridine hydrochloride) at high temperatures (e.g. 150° C. to 220° C.)). Such a reaction may be carried out in the presence of a suitable solvent, but preferably no further solvent is present.

Alternatively, compounds of formula X may be prepared from a compound of formula XIIB,

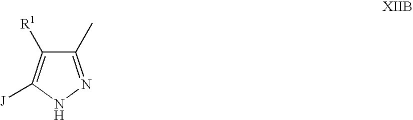

or a N-protected derivative thereof, wherein J and R¹ are as hereinbefore defined, using reagents and procedures known to those skilled in the art, for example such as those hereinbefore described in respect of preparation of compounds of formula I (process route (v)), or in respect of preparation of compounds of formula III (the process involving reaction with a compound of formula XI).

Compounds of formula XI (or a N-protected and/or O-protected (e.g. ester) derivative thereof) in which R¹ is as hereinbefore defined and preferably represents X or CF₃, may be prepared by reaction of a compound of formula XIII,

J—≡—R^{e} XIII

wherein R^{e} represents R¹ as hereinbefore defined and preferably, H or CF₃ and J is as hereinbefore defined, with a compound of formula XIV,

N₂—C(H)—C(O)OH XIV

or a O-protected (e.g. ester) derivative thereof, for example at elevated temperature (e.g. at between 80 and 120° C.) for between 1 and 3 days, optionally in the presence of an inert gas and preferably without the presence of solvent.

Compounds of formula XI (or a N-protected and/or O-protected (e.g. ester) derivative thereof) in which R¹ and J are as hereinbefore defined may be prepared by oxidation of a compound of formula XIIB as hereinbefore defined, under oxidation conditions known to those skilled in the art, for example such as those hereinbefore described in respect of preparation of compounds of formula III (the process involving reaction with a compound of formula X).

Alternatively, compounds of formula XI and XIIB (or, where applicable, a N-protected and/or O-protected (e.g. ester) derivative thereof) in which R¹ and J are as hereinbefore defined may be prepared by reaction of a compound of formula XIE, as hereinbefore defined, with an appropriate base (or a mixtures of bases), such as those listed in process (i) above, followed by quenching with an appropriate electrophile such as:
(a) for compounds of formula XI, a source of CO₂ (e.g. CO₂ gas; which addition is followed by the addition of a suitable proton source (e.g. HCl)), or a compound of formula XV,

R^{f}C(O)OL^{1e} XV
wherein R^{f} represents C₁₋₆ alkyl and L^{1e} represents a suitable leaving group such as halo (e.g. iodo, bromo or chloro); or
(b) for compounds of formula XIIB, a compound of formula XVI,

CH₃L^{1d} XVI
or the like (i.e. another suitable methylating reagent), wherein L^{1d} represents a suitable leaving group such as halo (e.g. iodo or bromo) or a sulfonate group (such as —OSO₂CF₃, OSO₂CH₃ and —OSO₂-aryl (e.g. —O-tosyl)).

Compounds of formula XIA may be prepared by reaction of 1-aminopyridinium iodide with a compound of formula XVII,

(R¹)(Cl)C═C(Cl)(R²) XVII

wherein R¹ and R² are as hereinbefore defined and the geometry of the double bond may be cis or trans, for example under conditions known to those skilled in the art (such as in the presence of a suitable base (e.g. potassium carbonate) and a suitable solvent (e.g. THF)). The skilled person will appreciate that the geometry around the double bond may effect the regioselectivity of the reaction.

Compounds of formula XIE may be prepared by reaction of a compound of formula XVII,

J—≡—R¹ XVIII

wherein R¹ and J are as defined hereinbefore, with diazomethane under conditions known to those skilled in the art, for example, in accordance with procedures described in T. Hanamoto et al., Chem. Commun, 2041 (2005), e.g. in the presence of a suitable solvent (e.g. hexane, diethyl ether, tetrahydrofuran or 1,4-dioxane or mixtures thereof) and optionally in the presence of an inert gas.

Compounds of formulae II, IV, V, VI, VIIIA, VIIIB, I, XIB, XIC, XID, XIIA, XII, XIV, XV, XVI, XVII and XVIII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituent X¹ and X² (if present) as hereinbefore defined may be modified one or more times, after or during the processes described above for preparation of compounds of formula I by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence. In the case where R¹ or R² represents a Cl or F group, such groups may be inter-converted (or converted from another halo group) one or more times, after or during the processes described above for the preparation of compounds of formula I. Appropriate reagents include NiCl₂ (for the conversion to a chloro group). The skilled person may also refer to “Comprehensive Organic Functional Group Transformations” by A. R. Katrity, O. Meth-Cohn and C. W. Rees, Pergamon Press, 1995.

Other transformations that may be mentioned include the conversion of a halo group (preferably iodo or bromo) to a cyano or 1-alkynyl group (e.g. by reaction with a compound which is a source of cyano anions (e.g. sodium, potassium, copper (I) or zinc cyanide) or with a 1-alkyne, as appropriate). The latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine). Further, amino groups and hydroxy groups may be introduced in accordance with standard conditions using reagents known to those skilled in the art.

Compounds of the invention may be isolated from their reaction mixtures using conventional techniques.

It will be appreciated by those skilled in the art that, in the processes described above and hereinafter, the functional groups of intermediate compounds may need to be protected by protecting groups. For example the pyrazole nitrogen may need to be protected. Suitable nitrogen-protecting groups include those which form:

(i) carbamate groups (i.e. alkoxy- or aryloxy-carbonyl groups);
(ii) amide groups (e.g. acetyl groups);
(iii) N-alkyl groups (e.g. benzyl or SEM groups);
(iv) N-sulfonyl groups (e.g. N-arylsulfonyl groups);
(v) N-phosphinyl and N-phosphoryl groups (e.g. diarylphosphinyl and diarylphosphoryl groups); or
(vi) N-silyl group (e.g. a N-trimethylsilyl group).

Further, the skilled person will appreciate that, in the case where there are two functional groups protected (e.g. in the case where the carboxylic acid group of the compound of formula III is an ester and the pyrazole nitrogen is protected with a benzenesulfonyl group, then both groups may be deprotected in one step (e.g. a hydrolysis step known to those skilled in the art).

Further protecting groups for the pyrazole nitrogen include a methyl group, which methyl group may be deprotected under standard conditions, such as employing a pyridine hydrochloride salt at elevated temperature, for example using microwave irradiation in a sealed vessel at 200° C.

The protection and deprotection of functional groups may tale place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3rd edition, T. W. Greene & P. G. M. Wutz, Wiley-Interscience (1999).

Compounds of the formula I and salts thereof are useful because they possess pharmacological activity. Such compounds/salts are therefore indicated as pharmaceuticals.

Certain compounds of formula I are novel per se.

The X¹- and X²-bearing phenyl group in compounds of formula I as hereinbefore defined may also be presented as follows:

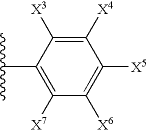

wherein the squiggly line dissecting the bond represents the point of attachment of the phenyl group to the rest of the compound of formula I, one of X³, X⁴, X⁵, X⁶ and X⁷ represents X¹ as hereinbefore defined and the others represent H or X² as hereinbefore defined.

According to a further aspect of the invention, there is provided a compound of formula I, as defined above, or a pharmaceutically-acceptable salt thereof, provided that:

(A) when R¹ represents Cl, R² represents H, and:
(1) X³, X⁴, X⁶ and X⁷ all represent H, then X⁵ does not represent Br, I or —C(O)CH₃;
(2) X³, X⁵, X⁶ and X⁷ all represent H, then X⁴ does not represent —C(O)CH₃;
(3) X³, X⁶ and X⁷ all represent H, then X⁴ does not represent Cl when X⁵ represents methyl or methoxy;
(4) X³, X⁵ and X⁷ all represent H, then X⁴ and X⁶ do not both represent —C(O)OCH₃ or —C(O)O-isopropyl;
(5) X⁴, X⁶ and X⁷ all represent H, then X⁵ does not represent F when X³ represents methyl;
(6) X³, X⁶ and X⁷ all represent H, then X¹ does not represent F when X⁴ represents —NO₂;
(7) X⁴, X⁵ and X⁶ represents H, then X⁷ does not represent isopropyl when X³ represents methyl;
(8) X³, X⁵ and X⁷ represents H, then X⁴ and X⁶ do not both represent methoxy;
(9) X⁴, X⁵, X⁶ and X⁷ all represent H, then X³ does not represent methoxy.
   (B) when R¹ represents H, R² represents CF₃, X⁴, X⁶ and X⁷ all represent H, then X³ does not represent chloro or CF₃ when X⁵ represents —NO₂.

According to a still further aspect of the invention, there is provided a compound of formula I, as defined above, or a pharmaceutically-acceptable salt thereof, with the additional provisos that, when R² represents CF₃ and:

(I) R¹ represents E or Cl, X⁷ represents H and:
(a) X⁴, X⁵ and X⁶ all represent H, then X³ does not represent CF₃;
(b) X⁴ and X⁶ both represent H, then X³ does not represent bromo when X⁵ represents —NO₂;
(C) X⁴ and X⁵ both represent H, then X³ does not represent chloro when X⁶ represents CF₃;
(d) X⁴ represents H, then X³ does not represent chloro when X⁵ represents —NO₂ and X⁶ represents chloro;
   (II) R¹ represents H or Cl, then X³, X, X⁵, X⁶ and X⁷ do not all represent F;
   (III) R¹ represents Cl and X⁴, X⁶ and X⁷ all represent H— then X³ does not represent chloro or CF₃ when X⁵ represents —NO₂;
   (IV) R¹ represents H, X³ represents Cl, then:
(i) X⁴, X⁵, X⁶ and X⁷ do not all represent H;
(ii) X⁴ does not represent Cl when X⁵ and X⁶ represent H or Cl and X⁷ represents H;
(iii) X⁵ does not represent Cl or Br when X⁴, X⁶ and X⁷ all represent H;
(iv) X⁷ does not represent Cl when Xs represents H, Cl or —NO₂ and X⁴ and X⁶ both represent H;
(v) X⁵ does not represent Cl when X⁶ represents Cl and X⁴ and X⁷ both represent H;
   (V) R¹ represents H and X³ represents Br, then Xs does not represent —OCF₃ when X⁴, X⁶ and X⁷ all represent H;
   (VI) R¹ represents H and X³ represents F or I, then X⁵ does not represent —NO₂ when X⁴, X⁶ and X⁷ all represent H;
   (VII) R¹ represents H and X³ represents —NO₂, then X⁵ does not represent C₁ or CF₃ when X⁴, X⁶ and X⁷ all represent H;
   (VIII) R¹ represents H, X³ represents CF₃, then X⁵ does not represent —NO₂ when X⁴ and X⁶ both represent H and X⁷ represents Cl;
   (IX) R¹ represents H, X³ represents CF₃, then X⁵ does not represent Cl, when 4, X⁶ and X⁷ all represent H.

Although compounds of formula I and salts thereof may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised), may therefore be described as “prodrugs” of compounds of formula I. All prodrugs of compounds of formula I are included within the scope of the invention.

By “prodrug of a compound of formula I”, we include compounds that form a compound of formula I, in an experimentally-detectable amount, within a predetermined time (e.g. about 1 hour), following oral or parenteral administration.

Compounds of formula I and salts thereof are useful because, in particular, they may inhibit the activity of lipoxygenases (and particularly 15-lipoxygenase), i.e. they prevent the action of 15-lipoxygenase or a complex of which the 15-lipoxygenase enzyme forms a part and/or may elicit a 15-lipoxygenase modulating effect, for example as may be demonstrated in the test described below. Compounds of the invention may thus be useful in the treatment of those conditions in which inhibition of a lipoxygenase, and particularly 15-lipoxygenase, is required.

Compounds of formula I are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Accordingly, compounds of formula I and salts thereof may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of formula I and salts thereof may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of formula I and salts thereof are indicated both in the therapeutic and/or prophylactic treatment of the above-mentioned conditions.

According to a further aspect of the present invention, there is provided a method of treatment of a disease which is associated with, and/or which can be modulated by inhibition of, a lipoxygenase (such as 15-lipoxygenase), and/or a method of treatment of a disease in which inhibition of the activity of a lipoxygenase, and particularly 15-lipoxygenase, is desired and/or required (e.g. inflammation), which method comprises administration of a therapeutically effective amount of a compound of formula I, as hereinbefore defined, or a pharmaceutically-acceptable salt thereof, to a patient suffering from, or susceptible to, such a condition

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of formula I and salts thereof will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of formula I and salts thereof may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of formula I, as hereinbefore defined, or a pharmaceutically-acceptable salt thereof, in admixture with a pharmaceutically acceptable adjuvant, diluent or carrier.

The invention further provides a process for the preparation of a pharmaceutical formulation, as hereinbefore defined, which process comprises bringing into association a compound of formula I as hereinbefore defined, or a pharmaceutically acceptable salt thereof with a pharmaceutically-acceptable adjuvant, diluent or carrier.

Compounds of formula I and salts thereof may also be combined with other therapeutic agents that are useful in the treatment of inflammation as defined herein (e.g. NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation):

According to a further aspect of the invention, there is provided a combination product comprising:
(A) a compound of formula I, as hereinbefore defined, or a pharmaceutically-acceptable salt thereof, and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of compound of the invention in conjunction with the other therapeutic agent, and may thus be presented either as separate formulations, wherein at least one of those formulations comprises compound of formula I or a salt thereof and at least one comprises the other therapeutic agent, or may be presented (i.e. formulated) as a combined preparation (i.e. presented as a single formulation including compound of the invention and the other therapeutic agent).

Thus, there is further provided:

(1) a pharmaceutical formulation including a compound of formula I, as hereinbefore defined, or a pharmaceutically-acceptable salt thereof, another therapeutic agent that is useful in the treatment of inflammation, and a pharmaceutically-acceptable adjuvant, diluent or carrier; and
(2) a kit of parts comprising components:
(a) a pharmaceutical formulation including a compound of formula I, as hereinbefore defined, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of formula I as hereinbefore defined, or a pharmaceutically acceptable salt thereof with the other therapeutic agent that is useful in the treatment of inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of formula I and pharmaceutically-acceptable salts thereof may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of formula I may have the advantage that they are effective and/or selective inhibitors of lipoxygenases, and particularly 15-lipoxygenase.

Compounds of formula I may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the stated indications or otherwise.

### Biological Test

The assay employed takes advantage of the ability of lipoxygenases to oxidize polyunsaturated fatty acids, containing a 1,4-cis-pentadiene configuration, to their corresponding hydroperoxy or hydroxyl derivatives. In this particular assay, the lipoxygenase was a purified human 15-lipoxygenase and the fatty acid was arachidonic acid. The assay is performed at room temperature (20-22° C.) and the following are added to each well in a 96-well microtiter plate:

a) 35 μL phosphate buffered saline (ABS) (pH 7.4);
b) inhibitor (i.e. compound) or vehicle (0.5 μl DMSO);
c) 10 μL of a 10× concentrated solution of 15-lipoxygenase in PBS. The plates are incubated for 5 minutes at room temperature;
d) 5 μl of 0.125 mM arachidonic acid in PBS. The plate is then incubated for 10 minutes at room temperature;
e) the enzymatic reaction is terminated by the addition of 100 μl MeOH; and
f) the amount of 15-hydroperoxy-eicosatetraenoic acid or 15-hydroxy-eicosatetraenoic acid is measured by reverse phase HPLC.

The invention is illustrated by way of the following examples, in which the following abbreviations may be employed:
aq. aqueous
BuLi n-butyllithium
DMAP 4-dimethylaminopyridine
DMF dimethylformamide
DIPEA diisopropylethylamine
EtOAc ethyl acetate
EtOH ethanol
MeOH methanol
MS mass spectrum
NMR nuclear magnetic resonance
rt room temperature
sat. saturated
TBTU O-benzotnazol-1-yl-N,N,N′,N′-tetramethyluronium tetrafluoroborate
THF tetrahydrofuran

Starting materials and chemical reagents specified in the syntheses described below are commercially available from, e.g. Sigma-Aldrich Pine Chemicals.

Unless otherwise stated, one or more tautomeric forms of compounds of the examples described hereinafter may be prepared in situ and/or isolated. All tautomeric forms of compounds of the examples described hereinafter should be considered to be disclosed.

### Synthesis of Intermediates:

### 1-Benzenesulfonyl-3-methylpyrazole (I)

A mixture of 3-methylpyrazole (5 g, 60.9 mmol), benzenesulfonyl chloride (8.55 mL, 67 mmol) and triethylamine (9.3 mL, 67 mmol) in acetonitrile was heated at reflux for 2 h, allowed to cool and concentrated. EtOAc (300 mL) was added and the solution was filtered and concentrated to provide a solid residue which was crystallised from EtOAc to give the title compound as an off-white powder (Yield: 7.92 g, 58%).

¹H-NMR (DMSO-d₆): δ 8.35 (d, 1H), 7.97-7.94 (m, 2H), 7.78 (tt, 1H), 7.66 (t, 2H), 6.43 (d, 1H), 2.17 (s, 3H).

### 5-Chloro-1-(2-chlorobenzenesulfonyl)-3-methylpyrazole (II)

BuLi (1.6M, 5.9 mL, 9.45 mmol) was added under argon to a solution of 1-benzenesulfonyl-3-methylpyrazole (940 mg, 4.5 mmol; see intermediate (I) above) in THF (50 mL) at −78° C. The mixture was stirred for approximately 30 min before hexachloroethane (3.7 g, 15.8 mmol) was added. After stirring at −78° C. for 18 h, NH₄Cl (aq, sat, 50 mL) was added and the mixture was allowed to come to rt. Water (50 mL) was added, the layers separated, and the aqueous phase extracted with EtOAc (2×100 mL). The combined organic phases were dried (Na₂SO₄) and concentrated. Purification by chromatography (1:4 EtOAc/heptane), followed by recrystallisation from EtOAc/heptane, gave the title compound as white crystals (Yield: 1.1 g, 84%).

¹H-NMR (DMSO-d₆): δ 8.17 (dd, 1H), 7.87-7.67 (m, 4H), 2.15 (s, 3H).

### 5-Chloro-3-methylpyrazole (III)

Sodium ethoxide (2.5M, 16.1 mL, 40.3 mmol) was added to a solution of 5-chloro-1-(2-chlorobenzenesulfonyl)-3-methylpyrazole (6.9 g, 27 mmol; see Intermediate (II) above) dissolved in EtOH (50 mL). The solution was stirred for 30 min at rt, water (100 mL) was added, the mixture was neutralised using HCl (aqueous, 2M) and extracted with EtOAc (3×100 mL). Concentration of the combined organic phases resulted in precipitation prior to complete solvent removal. The precipitate was filtered off and the filtrate was concentrated to give the title compound as a brown oil that crystallised on standing (Yield: 1.0 g, 33%) which was used without further purification.

¹H-NMR (DMSO-d₆): δ 12.66 (br s, 1H), 6.03 (d, 1H), 2.20 (s, 3H).

### 5-Chloro-3-methylpyrazole (III) (Alternative Synthesis)

A mixture of 5-chloro-1,3-dimethylpyrazole (7.00 g, 54 mmol) and pyridine hydrochloride (37.0 g, 320 mmol) was heated at 200° C. for 18 h. Hydrochloric acid (aq., 2M, 200 mL) was added after cooling to −60° C. and the mixture was extracted with EtOAc (3×100 mL). The combined organic extracts were washed with NaCl (sat., aq., 150 mL), dried Na₂SO₄) and concentrated in vacuo to give the product as white crystals (Yield 4.03 g, 64%).

MS (M⁺+H) m/z 117.

¹H NMR (DMSO-d₆, 400 MHz) δ 12.66 (s, 1H), 6.02 (s, 1H), 2.20 (s, 3H).

### 5-Chloropyrazole-3-carboxylic Acid (IV)

A solution of KMnO₄ (3.5 g, 22 mmol) in water (120 mL) was added in portions over a period of 5 h at 70° C. to a solution of 5-chloro-3-methylpyrazole (1.0 g, 8.8 mmol; see Intermediate (III) above) in water (50 mL) and tert-butanol (1 mL). The mixture was stirred at 70° C. overnight and filtered through Celite®. The colourless filtrate was concentrated and acidified with HCl (aq., 2M). Filtration gave the title compound as a white powder which was used without further purification. (Yield: 913 mg, 80%).

¹H-NMR (DMSO-d₆): δ 6.80 (s, 1M), 4.40 (br s, 1M).

### 1-Benzenesulfonylpyrazole (V)

A solution of pyrazole (5 g, 73 mmol), benzenesulfonyl chloride (8.5 mL, 67 mmol), and triethylamine (6.8 mL, 67 mmol) in acetonitrile (250 mL) was stirred for 30 min at reflux. The mixture was cooled and filtered. The filtrate was concentrated to a yellow residue that was purified by chromatography (1:4 EtOAc/heptane). Recrystallisation from EtOAc/heptane gave the title compound as colourless plates (Yield: 11.99 g, 86%).

¹H-NMR (DMSO-d₆): δ 8.48 (d, 1H), 7.97 (d, 2H), 7.90 (d, 1H), 7.79 (t, 1H), 7.67 (t, 2M), 7.61-7.60 (m, 1H).

### 5-Chloropyrazole (VI)

BuLi (1.6M, 3.4 mL, 5.4 mmol) was added under argon to a solution of 1-benzenesulfonylpyrazole (750 mg, 3.6 mmol; see Intermediate (V) above) in THF (50 mL) at −78° C. The mixture was stirred for 45 min before hexachloroethane (1.70 g, 7.2 mmol) was added in one portion. After stirring at −78° C. for 10 min, the mixture was allowed to warm to 10-15° C. over 75 min. The mixture was poured into H₂O Cl (1:1, aq, sat, 50 mL). The layers were separated and the aqueous phase was extracted with EtOAc (2×50 mL). The combined organic phases were dried (Na₂SO₄) and concentrated. The semi-solid residue was dissolved in MeOH (30 mL) followed by addition of sodium methoxide (30% in MeOH, 1.6 mL, 7.2 mmol). Stag at rt for 45 min, addition of NaHCO₃ (sat., aq., 1 mL) followed by extraction, concentration of the extract and purification of the residue by chromatography (1:1 EtOAc/heptane) gave the title compound as a white solid (Yield: 78 mg, 21%).

### 1-Benzenesulfonyl-3-chloropyrazole (VII)

A solution of 5-chloropyrazole (35 mg, 0.34 mmol; see Intermediate (VI) above), benzene sulfonylchloride (0.044 mL, 0.34 mmol), and triethylamine (0.047 mL, 0.34 mmol) in acetonitrile (250 mL) was stirred for 4 h at 60° C. The mixture was cooled and concentrated. Purification by chromatography (1:4 EtOAc/heptane) gave the title compound as colourless needles (Yield: 42 mg, 51%).

¹H-NMR (DMSO-d₆): δ 8.61 (d, 1H), 8.01 (d, 2H), 7.84 (t, 1H), 7.71 (t, 2H), 7.79 (d, 1H).

### 4,5-Dichloropyrazole-3-carboxylic Acid (VII)

Chlorine gas was bubbled slowly through a stirred solution of 5-chloropyrazole-3-carboxylic acid (3.00 g, 20.5 mmol; see intermediate (IV) above) in water (2.0 L) at rt over 3 h. The solution was stirred for 18 h in an open flask and then concentrated in vacuo. The resulting slurry was extracted with EtOAc (3×100 mL), the combined organic phases were washed with NaCl (sat., aq., 100 mL), dried (Na₂SO₄) and concentrated in vacuo to give the product as a white powder (Yield 3.20 g, 86%).

MS (M⁻−H) m/z 179.

¹H NMR DMSO-d₆, 400 MHz) δ 14.44 (s, 1H) 14.09 (s, 1H).

### 4,5-Bis(trifluoromethyl)pyrazole-3-carboxylic Acid (M)

### (a) 2,3-Bis(trifluoromethyl)pyrazolo[1,5-a]pyridine

A mixture of 1-aminopyridinium iodide (3.00 g, 13.51 mmol), K₂CO₃ (3.73 g, 27.02 mmol) and 2,3-dichloro-1,1,1,4,4,4-hexafluoro-but-2-ene (mixture of cis- and trans-isomers, 9.18 g, 39.41 mmol) were stirred in THF (100 mL) at rt for 24 h. The mixture was partitioned between EtOAc (100 mL), water (100 mL) and hydrochloric acid (2M, 5 mL), the phases were separated, the organic phase washed with NaCl (50 mL), dried. (Na₂SO₄) and concentrated in vacuo. The residue was dissolved in MeOH (25 mL) and filtered through Celite®. The filtrate was concentrated to give the title compound as slightly yellow needles (Yield: 2.95 g, 86%).

¹H NMR (CDCl₃, 400 MHz) δ 8.45 (ddd, 1H), 7.75 (dd, 1H), 7.39 (ddd, 1H), 7.02 (ddd, 1H).

### (b) 4,5-Bis(trifluoromethyl)pyrazole-3-carboxylic Acid

KMnO₄ (7.74 g, 49.0 mmol) was added portion-wise to a mixture of 2,3-bis(trifluoromethyl)pyrazolo[1,5-a]pyridine (2.49 g, 9.80 mmol), t-BuOH (30 mL) and water (120 mL). After stirring at rt for 24 h the mixture was filtered through Celite®. The filtrate was washed with CH₂Cl₂ (2×50 mL), then the pH was adjusted to 1 with concentrated hydrochloric acid (aq.). The mixture concentrated in vacuo to give a solid, which was extracted with acetone (3×20 mL). The combined extracts were concentrated and the residue crystallised from hydrochloric acid (aq., 0.1M; 2.5 mL). The solid was collected, washed with water (2×0.5 mL) and dried in vacuo to give the title compound as a white solid (Yield: 1.63 g, 67%).

¹³C NMR (CD₃OD, 100 MHz) δ 159.2 (s), 141.3 (q), 137.7 (s), 122.4 (q), 121.6 (q), 112.4 (q).

### 4-Trifluoromethylpyrazole-3-carboxylic Acid Ethyl Ester (X)

A solution of trimethylsilyldiazomethane in diethyl ether (2.0 M, 1.8 mL, 3.6 mmol) was added slowly under argon at 0° C. to a stirred solution of ethyl 4,4,4-trifluoro-2-butynate (0.50 g, 3.0 mmol) in diethyl ether (10 mL). The mixture was stirred at 0° C. for 5 min, the ice-bath was removed and the solution stirred at ambient temperature for 2 h. The mixture was concentrated and the residue purified by flash column chromatography (EtOAc/heptane) to give the product as a white powder (Yield: 465 mg, 75%).

¹H NMR (DMSO-d₆, 400 MHz) δ 14.1 (br. s, 1H), 8.49 (s, 1H), 4.30 (q, 2H), 1.28 (t, 3H).

### 4-Chloro-5-trifluoromethylpyrazole-3-carboxylic Acid (XI)

KMnO₄ (10.7 g, 67.7 mmol) was added portion-wise to a mixture of 5-trifluoromethyl-4-chloro-3-methylpyrazole (5.0 g, 27.1 mmol), t-BuOH (50 mL) and water (250 mL). The mixture was stirred at 75° C. for 3 days. After cooling to rt the precipitate was filtered off and the filtrate was concentrated in vacuo. Concentrated hydrochloric acid (aq., 10 mL) was added and the mixture extracted with EtOAc (5×30 mL). The combined extracts were washed with NaCl (sat., aq.; 50 mL), dried (Na₂SO₄) and concentrated in vacuo to give the product as a white solid (Yield 4.90 g, 84%).

MS (M⁻−H) m/z=213.

### 2-Amino-N-methylbenzenesulfonamide (XII)

### (a) N-Methyl-2-nitrobenzenesulfonamide

2-Nitrobenzenesulfonyl chloride (2.22 g, 10 mmol) was added portion-wise to a mixture of methylamine hydrochloride (810 mg, 12 mmol) and triethylamine (3.34 mL, 24 mmol) in CH₂Cl₂ (100 mL) at 0° C. The mixture was allowed to warm to rt, stirred for 1 h and then MeOH (20 mL) was added. The mixture was stirred for additional 1.5 h at rt and then CH₂Cl₂ (50 mL) was added. The mixture was washed with hydrochloric acid (aq., 1M, 100 mL) and NaCl (sat., aq., 50 mL), dried (Na₂SO₄) and concentrated in vacuo to give yellow needles. Recrystallisation from CH₂Cl₂₁MeOH gave the sub-title compound as slightly yellow needles (Yield 1.41 g, 65%).

¹H NMR (DMSO-d₆, 400 MHz) δ 8.00-7.95 (m, 2H), 7.95-7.86 (m, 3H), 2.54 (d, 3H).

### (b) 2-Amino-N-methylbenzenesulfonamide

N-Methyl-2-nitrobenzenesulfonamide (1.40 g, 6.47 mmol) in MeOH (30 mL) was hydrogenated over Pd on carbon (10%, 300 mg) at ambient temperature and pressure for 2.5 h. The mixture was filtered through Celite® and the filtrate concentrated in vacuo. The residue was purified by column chromatography (EtOAc/heptane) to give the title compound (1.10 g, 91%) as a colourless oil.

¹H NMR (DMSO-d₆, 400 MHz) δ 7.46 (dd, 1H), 7.33 (q, 1H), 7.26 (ddd, 1H), 6.81 (dd, 1H), 6.62 (ddd, 1H), 5.90 (s, 2H), 2.36 (d, 3H).

### 2-Amino-N,N-dimethylbenzenesulfonamide (XIII)

### (a) N,N-Dimethyl-2-nitrobenzenesulfonamide

Prepared by a procedure analogous to that described above for N-methyl-2-nitrobenzenesulfonamide using dimethylamine hydrochloride (978 mg, 12 mmol) instead of methylamine hydrochloride. Yield: 1.15 g (51%) of white needles.

¹H NMR (DMSO-d₆, 400 MHz): δ 8.00-7.83 (m, 4H), 2.82 (s, 6H).

### (b) 2-Amino-N,N-dimethylbenzenesulfonamide

Prepared by a procedure analogous to that described above for 2-amino-N-methylbenzenesulfonamide from N,N-dimethyl-2-nitrobenzenesulfonamide (1.15 g, 5.0 mmol) instead of N-methyl-2-nitrobenzenesulfonamide. Yield: 889 mg (89%) of an almost colourless solid.

¹H NMR (DMSO-d₆, 400 MHz). δ 7.39 (dd, 1H), 7.31 (ddd, 1H), 6.87 (dd, 1H), 6.65 (ddd, 1H), 6.05 (s, 2H), 2.64 (s, 6H).

### 5-Difluoromethyl-4-chloropyrazole-3-carboxylic Acid (XIV)

### (a) 5-Difluoromethylpyrazole-3-carboxylic Acid

KMnO₄ (2.74 g, 9.45 mmol) was added in portions to a mixture of 5-difluoromethyl-3-methylpyrazole (500 mg, 3.78 mmol), t-BuOH (10 mL) and water (100 mL). The mixture was stirred at 75° C. for 18 h. After cooling to rt the precipitate (MnO₂) was filtered off and the filtrate was concentrated. HCl (aq., conc.; 2.0 mL) was added and the mixture was extracted with EtOAc (5×20 mL). The combined extracts were washed with NaCl (sat., aq.; 25 mL), dried (Na₂SO₄) and concentrated. The material was purified using reverse phase column (RP-18) and CH₃CN/water (1:2) as eluent (Yield: 250 mg, 41%).

MS (M⁻−H) m/z=161.

¹H NMR (DMSO-d₆, 400 MHz) δ 14.27 (s, 1H), 13.60 (br. s, 1H), 7.03 (t, 1H), 6.97 (s, 1H).

### (b) 5-Difluoromethyl-4-chloropyrazole-3-carboxylic Acid

Chlorine gas was bubbled slowly through a stirred solution of 5-difluoro-methylpyrazole-3-carboxylic acid (100 mg, 0.62 mmol) in water (100 mL) at rt over 3 h. The solution was stirred for 18 h in an open flask and concentrated. The slurry was extracted with EtOAc (3×20 mL), the combined organic phases were washed with NaCl (sat., aq., 25 mL), dried (Na₂SO₄) and concentrated in vacuo to give the product as a white powder (Yield 106 mg, 87%).

MS (M⁻−H) m/z=195, 197.

### EXAMPLES

### Example 1

### 4-Chloro-N-(2-chloro-4-fluorophenyl)pyrazole-3-carboxamide

### (a) 4-Chloro-3-methylpyrazole Hydrochloride

A stirred solution of 3-methylpyrazole (50 mmol, 4.10 g) in carbon tetrachloride (50 mL) was saturated with chlorine gas at −78° C. The temperature was allowed to rise to rt and the mixture was stirred overnight. The slurry was diluted with pentane (50 mL) and stirred for an additional 30 min. The white crystalline solid was filtered off, washed with pentane (2×50 mL) and dried to provide the sub-title compound (Yield 7.50 g (98%)).

MS (M⁺+H) m/z=117.

¹H NMR (DMSO-d₆, 400 MHz) δ 13.38 (s, 2H), 7.68 (s, 1H), 2.16 (s, 3H).

¹³C NMR (DMSO-d₆, 100 MHz) δ 139.1, 132.2, 106.8, 9.3.

### (b) 4-Chloropyrazole-3-carboxylic acid

A well-stirred mixture of 4-chloro-3-methylpyrazole hydrochloride (20 mmol, 3.06 g; see step (a)) and potassium permanganate (50 mmol, 11.4 g) in water (500 mL) was stirred for 3 days at rt and then for 5 h at 70° C. The mixture was filtered and concentrated. Hydrochloric acid (aq., 1M; 50 mL) was added and the mixture was extracted with EtOAc (5×50 mL). The combined extracts were washed with NaCl (sat., aq.), dried Na₂SO₄) and concentrated to give 640 mg (22%) of the sub-title compound as a white solid.

¹H NMR (DMSO-d₆, 400 MHz) δ 13.47 (br s, 2H), 7.92 (br s, 1H).

### (c) 3,8-Dichlorodipyrazolo[1,5-a;1′,5′-d]pyrazine-4,9-dione

A mixture of 4-chloropyrazole-3-carboxylic acid (2.0 mmol, 300 mg; see step (b) above) in thionyl chloride (25 mL) was heated at reflux for 3 days. The excess thionyl chloride was removed in vacuo and the crude product employed in the next step without purification.

### (d) 4-Chloro-N-(2-chloro-4-fluorophenyl)pyrazole-3-carboxamide

A mixture of 3,8-dichlorodipyrazolo[1,5-a;1′,5′-d]pyrazine-4,9-dione (0.20 mmol, 51 mg) and 2-chloro-4-fluoroaniline (1.0 mmol, 146 mg) was heated at 120° C. for 1 h, cooled to ambient temperature and diluted with pentane (5 mL). The precipitate was filtered off and washed with pentane (30 mL). Crystallisation from EtOH:water (4:1, 20 mL) gave 84 mg (77%) of the title compound as a white solid.

MS (M⁺+H) m/z=274.

¹H NMR (DMSO-d₆, 400 MHz) δ 13.8 (br s, 1H), 9.65 (s, 1H), 820 (s, 1H), 7.96 (dd, 1H), 7.57 (dd, 1H), 7.28 (ddd, 1H).

### Example 2

### 5-Chloro-N-(2-chloro-4-fluorophenyl)pyrazole-3-carboxamide

### (a) 5-Chloro-3-methylpyrazole

A mixture of 5-chloro-1,3-dimethylpyrazole (2.6 mmol) and pyridine hydro-chloride (13.1 mmol) in a sealed 5 mL process vial was heated using microwave irradiation for 2 h at 200° C. After cooling to rt, EtOAc (15 mL) was added and the mixture was washed with HCl (aq., 2M; 10 mL), NaCl (sat, aq.), dried (MgSO₄) and concentrated to afford the sub-title compound as a white solid (Yield: 210 mg (67%)).

MS (M⁺+H) m/z=117.

¹H-NMR (DMSO-d₆, 400 MHz), δ 12.66 (br s, 1H), 6.03 (m, 1H), 2.19 (s, 3H).

### (b) 5-Chloropyrazole-3-carboxylic Acid

A mixture of 5-chloro-3-methylpyrazole (3.6 mmol; see step (a) above), water (6 mL) and tert-butanol (1.2 mL) was heated to 75° C., after which KMnO₄ (1.42 g, 9 mmol) was added. The mixture was stirred at 75° C. overnight and filtered hot. The solids were washed with boiling water. The combined cooled filtrates were extracted with EtOAc, and the combined extracts washed with NaCl (sat., aq.), dried (MgSO₄) and concentrated. The crude solid was recrystallised from EtOAc/hexane/pentane to give the sub-title compound as white crystals (Yield: 350 mg (67%)).

¹H-NMR (DMSO-d₆, 400 MHz), δ 13.65 (br s, 1H), 6.80 (s, 1H)

### (c) 5-Chloro-N-(2-chloro-4-fluorophenyl)pyrazole-3-carboxamide

A mixture of 5-chloropyrazole-3-carboxylic acid (1 mmol; see step (b) above) and SOCl₂ (1 in L) was heated at reflux for 18 h, cooled and concentrated. A portion of the resulting white solid (70 mg) was mixed with DMAP (0.27 mmol) and 2-chloro-4-fluoroaniline (0.27 mmol) in CH₂Cl₂ (10 mL) and stirred at 60° C. for 20 h. After cooling to rt, the solid was filtered off and washed with CH₂Cl₂. The solid was dissolved in EtOAc (15 mL) and washed with HCl (aq., 1M) and NaCl (sat, aq.). The organic phase was dried (MgSO₄) and concentrated. Crystallisation (EtOH/water) furnished the title compound as a white powder (Yield: 28.9 mg (39%)).

MS (M⁺+H) m/z=274.

¹H-NMR (DMSO-d₆, 400 MHz), δ 10.21 (br s, 1H), 7.58 (dd, 2H), 7.27-7.32 (m, 1H), 7.09 (br s, 1H).

### Example 3

### 5-Chloro-N-(2,4-dichlorophenyl)pyrazole-3-carboxamide

BuLi (1.6M, 0.116 mL, 0.19 mmol) was added under argon to a solution of 1-benzenesulfonyl-3-chloropyrazole (30 mg, 0.12 mmol; see Intermediate (VI) above) in THF (2 mL) at −78° C. The mixture was allowed to stir for 30 min before 2,4-dichlorophenylisocyanate (46 mg, 0.25 mmol) was added. The mixture was stirred at −78° C. for a further 18 h, after which NH₄Cl (aq, sat; 2 mL) and EtOAc (20 mL) was added. The layers were separated and the aqueous phase extracted with EtOAc (10 mL). The combined organic phases were dried (Na₂SO₄) and concentrated. Purification by chromatography (1:4 EtOAc/heptane) gave a white solid residue which was dissolved in MeOH (10 mL). Sodium methoxide (30% in MeOH, 0.024 mL, 0.1 mmol) was added and the mixture was stirred at rt for 3 days, after which NH₄Cl (sat., aq.; 20 mL) was added. The mixture was diluted with water (30 mL) and the EtOH removed in vacuo. The aqueous residue was extracted with EtOAc (3×50 mL) and the combined extracts were dried (Na₂SO₄) and concentrated. Chromatography (1:4 EtOAc/heptane) gave the title compound as a white solid (Yield: 7 mg (35%)).

¹H-NMR (DMSO-d₆): δ 13.4 (br s, 1H), 10.2 (s, 1H), 7.76 (s, 1H), 7.57 (s, 1H), 7.48 (dd, 1H), 7.10 (s, 1H).

### Example 4

### 3-Chloro-N-(2,3-dichlorophenyl)pyrazole-5-carboxamide

### (a) 2-Benzenesulfonyl-5-chloro-pyrazole-3-carboxylic Acid (2,3-dichloro-phenyl)-amide

1-Benzenesulfonyl-3-chloropyrazole (0.41 mmol; see Intermediate (Vat)) was dissolved in dry THF (10 mL) under argon at −78° C. BuLi (0.38 mL, 1.6M in hexane, 0.62 mmol) was added and the mixture was stirred for 45 min, after which 2,3-dichlorophenylisocyanate (116 mg, 0.62 mmol) was added. The mixture was stirred for 18 h at −78° C. NH₄Cl (sat, aq., 10 mL) was added and the mixture extracted with EtOAc (3×30 mL). The combined extracts were dried (Na₂SO₄) and concentrated. Purification by chromatography gave the sub-title compound (115 mg, 65%) as a white powder.

¹H-NMR (DMSO-d₆): 10.97 (s, 1H), 8.08 (d, 2H), 7.85 (t, 1H), 7.75-7.70 (m, 3H), 7.60 (d, 1H), 7.46 (t, 1H), 7.13 (s, 1H).

### (b) 5-Chloropyrazole-3-carboxylic Acid (2-3-dichlorophenyl)amide

2-Benzenesulfonyl-5-chloropyrazole-3-carboxylic acid (2,3-dichlorophenyl)-amide (88 mg, 0.20 mmol) was dissolved in EtOH (5 mL), after which sodium hydroxide (aq., 4M, 0.3 mmol; 77 μl) was added. The mixture was heated at 70° C. for 2 h and concentrated. NaCl (sat, aq.; 10 mL) was added and the mixture was extracted with EtOAc (3×10 mL). The combined organic extracts were dried (Na₂SO₄), filtered through Celite® and concentrated. Purification by chromatography gave the title compound (18 mg, 30%) as a white powder.

¹H-NMR (DMSO-d₆): 14.12 (s, 1H), 10.29 (s, 1H), 7.59 (d, 2H), 7.42 (t, 1H), 7.07 (s, 1H).

### Example 5

### 5-Chloro-N-(2,4-difluorophenyl)pyrazole-3-carboxamide

### (a) 2-Benzenesulfonyl-5-chloropyrazole-3-carboxylic Acid (2,4-difluoro-phenyl)-amide

The subtitle compound was prepared in accordance with the procedure described in Example 4(a) from 1-benzenesulfonyl-3-chloropyrazole (0.41 mmol; see Intermediate (VII)), BuLi (0.38 mL, 1.6M in hexane, 0.62 mmol) and 2,4-di-fluorophenylisocyanate (96 mg, 0.62 mmol). Yield: 91 mg, (55%).

¹H-NMR (DMSO-d₆): 10.93 (s, 1H), 8.07 (d, 2H), 7.89-7.82 (m, 2H), 7.75-7.70 (m, 2H), 7.42 (dt, 1H), 7.21-7.13 (m, 2H).

### (b) 5-Chloropyrazole-3-carboxylic acid (2,4-difluorophenyl)amide

The title compound was prepared in accordance with the procedure described in Example 4(b) from 2-benzenesulfonyl-5-chloropyrazole-3-carboxylic acid (2,4-difluorophenyl)amide (98 mg, 0.25 mmol). Yield: 36 mg, (56%).

¹H-NMR (DMSO-d₆): 8.61 (s, 1H), 7.90 (s, 1H), 7.53 (dd, 1H), 7.39 (d, 1H), 7.25 (s, 1H), 6.80 (s 1H).

### Example 6

### N-(2-Chloro-4-fluorophenyl)-4-fluoropyrazole-3-carboxamide

### (a) 4-Fluoropyrazole-3-carboxylic Acid Ethyl Ester

The sub-title compound was prepared from pyrazole-3-carboxylic acid ethyl ester in accordance with a literature procedure (R. Storer, et al., Nucleosides & Nucleotides 18, 203 (1999). A mixture (˜2:1) of sub-title compound and unreacted staring material was obtained and used without further purification.

### (b) 4-Fluoropyrazole-3-carboxylic Acid

Sodium hydroxide (aq., 2M, 18 mmol; 9 mL) was added to a solution of a mixture (˜2:1) of 4-fluoropyrazole-3-carboxylic acid ethyl ester and pyrazole-3-carboxylic acid ethyl ester (1.2 g, ˜8 mmol; see step (a) above) in dioxane (9 mL) at rt and was stirred for 16 h. A second portion of aqueous sodium hydroxide (2M, 18 mmol, 9 mL) was added and the mixture was sired for another 4 h. The mixture was acidified with HCl (aq., 2M, 20 ml), concentrated, stirred with MeOH (30 mL) and filtered. The filtrate was concentrated and the residue crystallised from HCl (aq., 0.01M) to give a mixture (˜3:1) of the sub-title compound and pyrazole-3-carboxylic acid as a white solid (Yield: 267 mg (˜2 mmol, ˜25%)). This mixture was employed without flier purification.

¹H-NMR (DMSO-d₆): δ 13.7-13.1 (br s, 1.3H), 7.9-7.7 (m, 1H), 7.73 (d, 0.3H), 6.70 (d, 0.3H).

### (c) N-(2-Chloro-4-fluorophenyl)-4-fluoropyrazole-3-carboxamide

A mixture (˜3:1) of 4-fluoropyrazole-3-carboxylic acid and pyrazole-3-carboxylic acid (85 mg, 0.69 mmol), TBTU (242 mg, 0.75 mmol), 2-chloro-4-fluoro-phenylamine (130 mg, 0.89 mmol), and DIPEA (239 μL, 1.37 mmol) in DMF (2.5 mL) was stirred at rt for 3 days and at 85° C. for 16 h. TBTU (36 mg, 0.10 mmol) was added and the mixture was stirred for 1 hour at 85° C. The mixture was cooled and water (10 mL) and NaCl (sat, aq.; 10 mL) were added. The mixture was extracted with EtOAc (5×20 mL). The combined extracts were dried (Na₂SO₄), concentrated and purified by chromatography to give the title compound as a mixture (˜10:1) with pyrazole-3-carboxylic acid (2-chloro-4-fluorophenyl)amide. ¹H-NMR (DMSO-d₆): δ 13.47 (s, 1H), 9.56 (s, 1H), 8.00 (d, 1H), 7.99-7.86 (m, 1H), 7.55 (dd, 1H), 7.27 (ddd, 1H).

### Example 7

### 5-Chloro-N-(4-fluorophenyl)pyrazole-3-carboxamide

BuLi (1.6M, 0.38 mL, 0.62 mmol) was added under argon to a solution of 1-benzenesulfonyl-3-chloropyrazole (100 mg, 0.41 mmol; see Intermediate (VII)) in THF (10 mL) at −78° C. The mixture was stirred for 10 min before 4-fluoro-phenylisocyanate (0.071 mL, 0.62 mmol) was added. Stirring was continued at −78° C. for 18 h, after which NH₄Cl (sat., aq.; 6 mL), water and finally EtOAc were added. The phases were separated and the aqueous phase extracted with EtOAc. The combined extracts were concentrated to provide a brown oil, which crystallised on standing. The solid was dissolved in EtOH (10 mL) and sodium hydroxide (aq., 4M, 0.62 mmol; 0.15 mL) was added. The mixture was stirred at rt for 20 min before NH₄Cl (sat., aq.; 6 mL) was added. The mixture was diluted with water (15 mL) and the EtOH removed in vacuo. The aqueous phase was extracted with EtOAc (3×50 mL) and the combined organic phases were dried (Na₂SO₄) and concentrated. Purification by chromatography (1:4 EtOAc/heptane) gave the title compound as a white solid (Yield: 49 mg (50%)).

¹H-NMR (DMSO-d₆): δ 13.99 (br s, 1H), 10.26 (s, 1H), 7.73-7.69 (m, 2H), 7.20 (t, 2H), 7.07 (br s, 1H).

### Example 8

### 5-Chloro-N-(4-chlorophenyl)pyrazole-3-carboxamide

### (a) 2-Benzenesulfonyl-5-chloropyrazole-3-carboxylic acid (4-chlorophenyl)-amide

The sub-title compound was prepared in accordance with the procedure described in Example 4(a) from 1-benzenesulfonyl-3-chloropyrazole (100 mg, 0.41 mmol; see Intermediate (VII)), BuLi (0.38 mL, 1.6M in hexane, 0.62 mmol) and 4-chlorophenylisocyanate (105 mg, 0.62 mmol).

¹H-NMR (DMSO-d₆): 12.7 (s, 1H), 8.01-7.94 (m, 4H), 7.83 (t, 1H), 7.70 (t, 2H), 7.56 (d, 2H), 6.98 (s, 1H).

### (b) 5-Chloropyrazole-3-carboxylic acid (4-chlorophenyl)amide

The title compound was prepared in accordance with the procedure described in Example 4(b) from 2-benzenesulfonyl-5-chloropyrazole-3-carboxylic acid (4-chlorophenyl)amide (123 mg, 0.30 mmol). Yield: 12 mg (15%)

¹H-NMR (DMSO-d₆): δ 13.68 (s, 1H), 11.67 (s, 1H), 7.77 (d, 2H), 7.50 (d, 2H), 7.00 (s, 1H).

### Example 9

### N-(2-chloro-4-fluorophenyl)-5-(trifluoromethyl)pyrazole-3-carboxamide

### (a) 111-Trifluoro-4-methoxypent-3-en-2-one

A mixture of 2-methoxypropene (7.7 g, 132 mmol) and pyridine (9.7 mL, 120 mmol) was added drop-wise to trifluoroacetic acid anhydride (25.2 g, 120 mmol) while cooled at −30° C. Diethyl ether (50 mL) was added and the mixture was left for 18 h at rt. Filtration and concentration gave a yellow oil that was taken up in CH₂Cl₂. The mixture was washed with HCl (aq., 0.1M; 50 mL), water (50 mL), dried (Na₂SO₄) and concentrated affording 23 g of an orange oil which was used in the following step without any further purification.

¹H NMR (CDCl₃) δ 5.68 (s, 1H), 3.80 (s, 3M), 2.41 (s, 3H).

### (b) 3-methyl-5-trifluoromethylpyrazole

Hydrazine hydrate (4.0 g, 79 mmol) was added dropwise to a solution of 1,1,1-trifluoro-4-methoxypent-3-en-2-one (10 g, 59 mmol; see step (a) above) in EtOH (30 mL). The mixture was heated at reflux for 2 h, cooled and concentrated. The residue was taken up in diisopropyl ether and dried (Na₂SO₄). Concentration gave the sub-title compound that was used in the following step without further purification. Yield: 7.0 g (79%).

¹H-NMR (CDCl₃) δ6.15 (s, 1H), 2.29 (s, 3H).

### (c) 5-Trifluoromethylpyrazole-3-carboxylic Acid

A mixture of 3-methyl-5-trifluoromethylpyrazole (3.0 g, 20 mmol; see step (b) above) and KMnO₄ (3.0 g) in water (80 mL) was heated at 80° C. for 18 h. The mixture was filtered through Celite®. The filtrate was acidified with HCl (2M aqueous) and extracted with diethyl ether (3×50 mL). The combined extracts were dried (Na₂SO₄) and concentrated. The resulting compound (yellow crystals) was used without further purification. Yield: 1.6 g (44%).

### (d) N-(2-Chloro-4-fluorophenyl)-5-(trifluoromethyl)pyrazole-3-carboxamide

A solution of 5-trifluoromethylpyrazole-3-carboxylic acid (200 mg, 1.1 mmol; see step (c) above), 2-chloro-4-fluoroaniline (189 mg, 1.3 mmol) and DIPEA (285 mg, 2.2 mmol) in DMF (10 mL) was added TBTU (417 mg, 1.3 mmol). The mixture was left at rt for 18 h followed by addition of water (50 mL) and extraction with EtOAc (3×30 mL). The combined extracts were washed with water (50 mL), dried (Na₂SO₄) and concentrated. Purification by chromatography (EtOAc/Hept 1:10 to 1:1) gave the title compound as a colourless solid. Yield: 12 mg (4%).

¹H-NMR (DMSO-d₆) δ 14.69 (s, 1H), 10.33 (s, 1H), 7.60 (dd, 2H), 7.50 (br s, 1H), 7.31 (dt, 2M).

### Examples 10-29

### General Procedures

### Method A

A mixture of the relevant substituted pyrazole-3-carboxylic acid (intermediate VIII, 1.2 mmol) and SOCl₂ (10 mL) was stirred at 80° C. for 18 h. After cooling to rt the mixture was concentrated and the residue was dried. A mixture of the relevant arylamine (3.6 mmol) and CH₂Cl₂ (10 mL) was added to the residue. The mixture was stirred at 0.60° C. for 18 h. After cooling to rt the mixture was concentrated and the residue acidified with HCl (aq., 1M; 10 mL). The mixture was extracted with EtOAc (4×10 mL), the combined organic phases were then washed with NaCl (sat., aq.; 20 mL), dried (Na₂SO₄) and concentrated in vacuo. The residue was recrystallized from EtOH/water (1:1) and EtOAc/hexane (2:1).

### Method B

A mixture of the relevant substituted pyrazole-3-carboxylic acid (intermediate XI or XIV, 1.2 mmol) and SOCl₂ (10 mL) was stirred at 80° C. for 18 h. After cooling to rt the mixture was concentrated and the residue was dried in vacuo. A mixture of the relevant arylamine (2.4 mmol), DMAP (1.6 mmol) and CH₂Cl₂ (10 mL) was added to the residue. The mixture was stirred at 60° C. for 18 h. After cooling to rt the mixture was concentrated and the residue acidified with HCl (aq., 1M; 10 mL). The mixture was extracted with EtOAc (4×10 mL), the combined organic phases were washed with NaCl (sat., aq.; 20 mL), dried (Na₂SO₄) and concentrated. The residue was recrystallised from EtOH/water (1:1) and EtOAc/hexane (2:1).

### Method C

A mixture of TBTU (642 mg, 2.0 mmol), the relevant substituted pyrazole-3-carboxylic acid (intermediate IV, 1.0 mmol), the relevant arylamine (1.0 mmol), DIPEA (348 μL, 2.0 mmol) and DMAP (12 mg, 0.1 mmol) in dry DMF (5 mL) was stirred at 80° C. for 3 days. After cooling to rt the mixture was concentrated and the residue acidified with HCl (aq., 1M; 10 mL). The mixture was extracted with EtOAc (4×10 mL), the combined organic phases were then washed with NaCl (sat., aq.; 20 mL), dried (Na₂SO₄) and concentrated. The residue was purified by column chromatography (EtOAc/heptane).

### Method D

Trimethylaluminium (0.63 mL, 2.0M in hexanes, 1.25 mmol) was added to a solution of the relevant arylamine (0.50 mmol) in CH₂Cl₂ (2 mL) under argon at 0° C. A solution of the relevant substituted pyrazole-3-carboxylic acid ester (intermediate X, 0.25 mmol) in CH₂Cl₂ (2 mL) was added and the mixture was allowed to warm to rt. The mixture was sired at rt for 24 h and poured into HCl (aq., 0.01M; 10 mL). The pH was adjusted to −3 by dropwise addition of HCl (aq., 2M). The mixture was extracted with EtOAc (3×25 mL), the combined organic phases were washed with NaCl (sat, aq.; 30 mL), dried (Na₂SO₄) and concentrated. The residue was purified by chromatography (EtOAc/heptane) and recrystallised from ethyl acetate/heptane.

### Method E

Sodium hydride (60% in mineral oil, 60 mg, 1.5 mmol) was added to a solution of the relevant arylamine (1 mmol) in DMF (2 mL) at rt. The mixture was stirred for 5 min, then a solution of the relevant substituted pyrazole-3-carboxylic acid ester (intermediate X, 0.5 mmol) in DMF (2 mL) was added and the mixture was stirred at rt for 15 h. The mixture was poured into NaHCO₃ (sat, aq.; 15 mL) and extracted with EtOAc (3×20 mL). The combined extracts were washed with NaCl (sat., aq.; 20 mL), dried (Na₂SO₄) and concentrated. The crude product was purified by chromatography EtOAc/heptane).

### Method F

A mixture of the relevant substituted pyrazole-3-carboxylic acid (intermediate IV or IX, 1.0 mmol) and SOCl₂ (10 mL) was stirred at 80° C. for 18 h. After cooling to rt the mixture was concentrated and the residue dried in vacuo. A mixture of the relevant arylamine (1.0 mmol), DMAP (12 mg, 0.10 mmol), DMF (0.5 mL) and pyridine (1 mL) was added. The mixture was stirred at 80° C. for 21 h and concentrated in vacuo. The residue was purified by chromatography (EtOAc/heptane).

TABLE 1
Examples (Ex.) 10 to 29

| | | Prepared from | | | |
|---|---|---|---|---|---|
| | | Intermediate | | | |
| Ex. | Chemical name | No. | aniline | Method | Yield % |
| 10 | N-(2-Chloro-4-fluoro- | VIII | 2-Chloro-4- | A | 80 |
| | phenyl)-4,5-dichloro- | | fluoroaniline | | |
| | pyrazole-3-carboxamide | | | | |
| 11 | 4,5-Dichloro-N-(4-fluoro- | VIII | 4- | A | 49 |
| | phenyl)pyrazole-3- | | Fluoroaniline | | |
| | carboxamide | | | | |
| 12 | 4,5-Dichloro-N-(2,4- | VIII | 2,4- | A | 66 |
| | difluorophenyl)pyrazole- | | Difluoroaniline | | |
| | 3-carboxamide | | | | |
| 13 | N-(4-Chlorophenyl)-4,5- | VIII | 4- | A | 38 |
| | dichloropyrazole-3- | | Chloroaniline | | |
| | carboxamide | | | | |
| 14 | 4,5-Dichloro-N-(2- | VIII | 2-Trifluoro- | A | 56 |
| | trifluoromethoxyphenyl)- | | methoxyaniline | | |
| | pyrazole-3-carboxamide | | | | |
| 15 | 4-Chloro-N-(2-chloro-4- | XI | 2-Chloro-4- | B | 46 |
| | fluorophenyl)-5-trifluoro- | | fluoroaniline | | |
| | methylpyrazole-3- | | | | |
| | carboxamide | | | | |
| 16 | 4-Chloro-N-(4-fluoro- | XI | 4- | B | 72 |
| | phenyl)-5-trifluoromethyl- | | Fluoroaniline | | |
| | pyrazole-3-carboxamide | | | | |
| 17 | 4-Chloro-N-(2,4-difluoro- | XI | 2,4-Difluoro- | B | 86 |
| | phenyl)-5-trifluoromethyl- | | aniline | | |
| | pyrazole-3-carboxamide | | | | |
| 18 | 4-Chloro-N-(4-chloro- | XI | 4- | B | 95 |
| | phenyl)-5-trifluoromethyl- | | Chloroaniline | | |
| | pyrazole-3-carboxamide | | | | |
| 19 | 5-Chloro-N-(2-difluoro- | IV | 2-Difluoro- | C | 42 |
| | methoxyphenyl)pyrazole- | | methoxyaniline | | |
| | 3-carboxamide | | | | |
| 20 | 5-Chloro-N-(2-trifluoro- | IV | 2-Trifluoro- | C | 37 |
| | methoxyphenyl)pyrazole- | | methoxyaniline | | |
| | 3-carboxamide | | | | |
| 21 | N-(2-Chloro-4-fluoro- | X | 2-Chloro-4- | D | 21 |
| | phenyl)-4-trifluoromethyl- | | fluoroaniline | | |
| | pyrazole-3-carboxamide | | | | |
| 22 | N-(2,4-Dichlorophenyl)-4- | X | 2,4-Dichloro- | E | 48 |
| | trifluoromethylpyrazole- | | aniline | | |
| | 3-carboxamide | | | | |
| 23 | N-(4-Fluorophenyl)-4- | X | 4- | E | 15 |
| | trifluoromethylpyrazole- | | Fluoroaniline | | |
| | 3-carboxamide | | | | |
| 24 | N-(2-Chloro-4-fluoro- | IX | 2-Chloro-4- | F | 49 |
| | phenyl)-4,5-bis(trifluoro- | | fluoroaniline | | |
| | methyl)pyrazole-3- | | | | |
| | carboxamide | | | | |
| 25 | 5-Chloro-N-(2-chloro-4- | IV | 2-Chloro-4- | C | 24 |
| | isopropylphenyl)pyrazole- | | iso- | | |
| | 3-carboxamide | | propylaniline | | |
| 26 | 5-Chloro-N-(2-(N-methyl- | IV | 2-Amino-N- | F | 58 |
| | sulfamoyl)phenyl)pyrazole- | | methylbenzene- | | |
| | 3-carboxamide | | sulfonamide | | |
| | | | (intermediate XII) | | |
| 27 | 5-Chloro-N-(2-(N,N- | IV | 2-Amino-N,N- | F | 10 |
| | dimethylsulfamoyl)phenyl) | | dimethylbenzene- | | |
| | pyrazole-3-carboxamide | | sulfonamide | | |
| | | | (intermediate XIII) | | |
| 28 | 4-Chloro-5- | XIV | 4- | B | 8 |
| | difluoromethyl-N- | | Fluoroaniline | | |
| | (4-fluorophenyl)pyrazole- | | | | |
| | 3-carboxamide | | | | |
| 29 | 4-Chloro-N-(2-chloro-4- | XIV | 2-Chloro-4- | B | 7 |
| | fluorophenyl)-5- | | fluoroaniline | | |
| | difluoromethylpyrazole-3- | | | | |
| | carboxamide | | | | |

TABLE 2
Physical properties of the compounds of Examples 10-29

| | | MS | |
|---|---|---|---|
| | | (M⁻-H), | |
| Ex. | M.W. | m/z | ¹H NMR (DMSO-d₆, 400 MHz), δ |
| 10 | 310.54 | 306 | 14.54 (s, 1H), 9.73 (s, 1H), 7.87 (dd, 1H), |
| | | | 7.59 (dd, 1H), 7.30 (ddd, 1H) |
| 11 | 276.09 | 274 | 14.38 (br. s, 1H), 10.35 (s, 1H), 7.70-7.73 |
| | | | (m, 2H), 7.18-7.24 (m, 2H) |
| 12 | 294.08 | 292 | 14.47 (br. s, 1H), 9.94 (s, 1H), 7.71-7.72 |
| | | | (m, 1H), 7.36-7.41 (m, 1H), 7.11-7.16 (m, 1H) |
| 13 | 292.55 | 288 | 14.40 (br. s, 1H), 10.43 (s, 1H), 7.75-7.70 |
| | | | (m, 2H), 7.41-7.45 (m, 2H) |
| 14 | 342.10 | 338 | 14.54 (br. s, 1H), 9.78 (s, 1H), 7.96 (d, 1H), |
| | | | 7.43-7.49 (m, 2H), 7.33-7.38 (m, 1H) |
| 15 | 344.09 | 340 | 15.06 (br. s, 1H), 9.93 (s, 1H), 7.81-7.84 |
| | | | (m, 1H), 7.59-7.61 (dd, 1H), 7.29-7.34 (m, 1H) |
| 16 | 309.65 | 306 | 14.95 (br. s, 1H), 10.50 (s, 1H), 7.69-7.73 |
| | | | (m, 2H), 7.21-7.26 (m, 2H) |
| 17 | 327.64 | 324 | 15.01 (br. s, 1H), 9.96 (s, 1H), 7.73-7.78 |
| | | | (m, 1H), 7.37-7.43 (m, 1H), 7.12-7.17 (m, 1H) |
| 18 | 324.09 | 322 | 14.92 (br. s, 1H), 10.60 (s, 1H), 7.71 (d, 2H), |
| | | | 7.43-7.47 (m, 2H) |
| 19 | 289.67 | 286 | 14.13-13.88 (br. s, 1H), 10.13-9.93 (br. s, |
| | | | 1H), 7.69-7.54 (m, 1H), 7.38-7.25 (m, 3H), |
| | | | 7.15 (dd, 1H), 7.14-7.24 (m, 1H) |
| 20 | 307.66 | 304 | 14.17-13.92 (br. s, 1H), 10.37-10.19 (br. s, |
| | | | 1H), 7.74-7.57 (m, 1H), 7.53-7.34 (m, 4H), |
| | | | 7.10 (s, 1H) |
| 21 | 309.65 | 306 | 14.13 (s, 1H), 9.83 (s, 1H), 8.57 (s, 1H), |
| | | | 7.91 (dd, 1H), 7.58 (dd, 1H), 7.28 (ddd, 1H) |
| 22 | 326.10 | 322 | 14.17 (s, 1H), 9.81 (s, 1H), 8.59 (s, 1H), |
| | | | 8.04 (d, 1H), 7.75 (d, 1H), 7.48 (dd, 1H) |
| 23 | 275.20 | 272 | 14.04 (s, 1H), 10.37 (s, 1H), 8.52 (s, 1H), |
| | | | 7.81 (dd, 2H), 7.17 (dd, 2H) |
| 24 | 377.65 | 374 | 5.64-14.88 (br. s, 1H), 10.89-10.61 (br. s, 1H), |
| | | | 7.67 (dd, 1H), 7.60 (dd, 1H), 7.32 (ddd, 1H) |
| 25 | 300.18 | 296 | 14.06 (br. s, 1H), 10.04 (br. s, 1H), 7.49 |
| | | | (br. s, 1H), 7.44 (d, 1H), 7.07 (s, 1H), |
| | | | 3.00-2.82 (m, 1H), 1.24 (s, 3H), 1.21 (s, 3H) |
| 26 | 317.77 | 313 | 14.26 (br. s, 1H), 10.40 (br. s, 1H), 8.40 |
| | | | (br. s, 1H), 7.90-7.78 (m, 2H), 7.70 (dd, 1H), |
| | | | 7.38 (br. s, 1H), 6.88 (s, 1H), 2.45 (s, 3H) |
| 27 | 330.79 | 327 | 14.31 (br. s, 1H), 10.55 (br. s, 1H), 8.41 |
| | | | (br. s, 1H), 7.75 (d, 1H), 7.76 (dd, 1H), 7.42 |
| | | | (dd, 1H), 6.93 (s, 1H), 2.66 (s, 6H) |
| 28 | 289.64 | 288 | 14.56 (br. s, 1H), 10.39 (s, 1H), 7.76-7.72 |
| | | | (m, 2H), 7.33-7.08 (m, 3H) |
| 29 | 24.09 | 322 | 14.70 (br. s, 1H), 9.80 (s, 1H), 7.86 (dd, 1H), |
| | | | 7.58 (dd, 1H), 7.32 (ddd, 1H), 7.21 (t, 1H) |

### Example 30

Title compounds of the Examples were tested in the biological test described above and were found to exhibit an IC₅₀ of 10 μM or below. For example, the following representative compounds of the examples exhibited the following IC₅₀ values:

| | |
|---|---|
| Example 1: | 85 nM |
| Example 5: | 265 nM |
| Example 6: | 114 nM |
| Example 7: | 182 nM |
| Example 8: | 78 nM |
| Example 19: | 69 nM |

1. A compound of formula I, 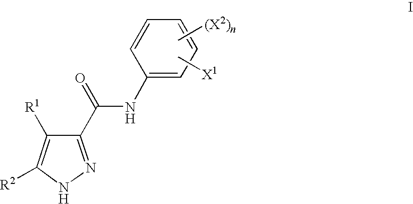
wherein,
R¹ and R² independently represent H, Cl, F, CHF₂ or CF₃, provided that at least one of R¹ and R² does not represent H;
X¹ represents halo, —R^{3a}, —OR^{3q} or —S(O)₂N(R^{4j})R^{5j};
X² represents halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —S(O)₂N(R^{4h})R^{5h}, —S(O)₂OH, —N(R^{3k})S(O)₂R^{3m}, —OC(O)R³ⁿ, —OC(O)OR^{3p} or —P(O)(OR⁴ⁱ)(OR⁵ⁱ);
n represents 0, 1, 2, 3 or 4;
m represents 0, 1 or 2;
R^{3a} represents C₁₋₆ alkyl optionally substituted by one or more substituents selected from F, Cl, —N(R^{4b})R^{5b}, —N₃, ═O and —OR^{3h};
R^{3b} to R^{3h}, R^{3k}, R³ⁿ, R^{3q}, R^{4a} to R^{4j}, R^{5a}, R^{5b}, R^{5d} and R^{5f} to R^{5j} independently represent hydrogen or C₁₋₆ alkyl optionally substituted by one or more substituents selected from F, Cl, —OCH₃, —OCH₂CH₃, —OCHF₂, and —OCF₃; or
any of the pairs R^{4a} and R^{5a}, R^{4b} and R^{5b}, R^{4d} and R^{5d}, R^{4f} and R^{5f}, R^{4g} and R^{5g}, R^{4f} and R^{5h}, and R^{4j} and R^{5j}, may be linked together to form a 3- to 6-membered ring, which ring optionally contains a further heteroatom in addition to the nitrogen atom to which these substituents are necessarily attached, and which ring is optionally substituted by ═O and/or C₁₋₆ alkyl, which alkyl group is optionally substituted by one or more F atom;
R³ⁱ, R^{3j}, R^{3m} and R^{3p} independently represent C₁₋₆ alkyl optionally substituted by one or more substituents selected from F, Cl, —OCH₃, —OCH₂CH₃, —OCHF₂, and —OCF₃,
or a pharmaceutically-acceptable salt thereof, for use as a pharmaceutical.

2. A compound as claimed in claim 1, wherein X¹ represents halo or —R³.

3. A compound as claimed in claim 1 or claim 2, wherein R¹ and R² independently represent H, F or Cl.

4. A compound as claimed in claim 1, wherein n is 0 or 1.

5. A compound as claimed in claim 4, wherein n is 1.

6. A compound as claimed in claim 1, wherein, when any of the pairs R^{4a} and R^{5a}, R^{4b} and R^{5b}, R^{4d} and R^{5d}, R^{4f} and R^{5f}, R^{4g} and R^{5g}, R^{4h} and R^{5h}, and R^{4j} and R^{5j}, are linked together, they form a 5- to 6-membered ring, which ring optionally contains a further heteroatom and is optionally substituted by methyl, —CHF₂ or CF₃.

7. A compound as claimed in claim 1, wherein X¹ represents —OR³, F, Cl or R^{3a}.

8. A compound as claimed in claim 7 wherein X¹ represents F, Cl or C₁₋₃ alkyl optionally substituted by one or more fluoro atoms.

9. A compound as claimed in claim 8, wherein X¹ represents F, Cl, CH₃ or CF₃.

10. A compound as claimed in claim 1, wherein X² represents F, Cl, Br, —R^{3a}, —CN, —C(O)R^{3b}, C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j} or —S(O)₂N(R^{4h})R^{5h}.

11. A compound as claimed in claim 10, wherein X² represents —CN, —C(O)N(R^{4a})R^{5a}, N(R^{4b})R^{5b}, —N(H)C(O)R^{4c}, —S(O)₂CH₃, —S(O)₂CF₃—S(O)₂N(R^{4h})R^{5h}, F, Cl, —R^{3a} or OR^{3h}.

12. A compound as claimed in claim 1, wherein R^{3a} represents C₁₋₆ alkyl optionally substituted by one or more substituents selected from F and —OR^{3h}.

13. A compound as claimed in claim 12, wherein R^{3a} represents C₁₋₄ alkyl optionally substituted by one or more F atoms.

14. A compound as claimed in claim 1, wherein R^{3b}, R^{3c}, R^{3h}, R^{4a} to R^{4h}, R^{4j}, R^{5a}, R^{5b}, R^{5d}, R^{5f} to R^{5h} and R^{5j} independently represent hydrogen or C₁₋₄ alkyl, or the relevant pairs are linked together.

15. A compound as claimed in claim 1, wherein R^{3d} to R^{3g} independently represent C₁₋₂ alkyl or hydrogen.

16. A compound as claimed in claim 1, wherein R³ and R^{3j} independently represent C₁₋₄ alkyl optionally substituted by one or more F atoms.

17. A compound as claimed in claim 1, wherein R^{3h} represents hydrogen or C₁₋₄ alkyl optionally substituted by one or more fluoro atoms.

18. A compound as claimed in any one of the preceding claims claim 1, wherein R^{4a}, R^{4b}, R^{4c}, R^{4h}, R^{5a}, R^{5b} and R^{5h} independently represent hydrogen, methyl or ethyl, or the relevant pairs are linked together to form a pyrrolidinyl, piperidinyl, morpholinyl or a 4-methylpiperazinyl ring.

19. A pharmaceutical formulation including a compound as defined in claim 1, or a pharmaceutically acceptable salt thereof, in admixture with a pharmaceutically acceptable adjuvant, diluent or carrier.

20. (canceled)

21. A method as claimed in claim 24 wherein the lipoxygenase is 15-lipoxygenase.

22. A method as claimed in claim 21, wherein the disease is inflammation and/or has an inflammatory component.

23. A method as claimed in claim 22 wherein the inflammatory disease is asthma, chronic obstructive pulmonary disease, pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease or another malignancy.

24. A method of treatment of a disease in which inhibition of the activity of a lipoxygenase is desired and/or required, which method comprises administration of a therapeutically effective amount of a compound of formula I as defined in claim 1, or a pharmaceutically-acceptable salt thereof, to a patient suffering from, or susceptible to, such a condition.

25. A compound of formula Ia, 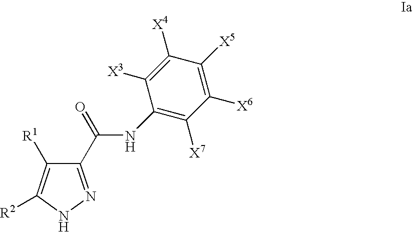
wherein one of X³, X⁴, X⁵, X⁶ and X⁷ represents X¹ and the others represent H or X², and X¹, X², R¹ and R² are as defined in claim 1, or a pharmaceutically-acceptable salt thereof, provided that:
(A) when R¹ represents Cl, R² represents H, and:
(1) X³, X⁴, X⁶ and X⁷ all represent H, then X⁵ does not represent Br, I or —C(O)CH₃;
(2) X³, X⁵, X⁶ and X⁷ all represent H, then X⁴ does not represent —C(O)CH₃;
(3) X³, X⁶ and X⁷ all represent H, then X⁴ does not represent Cl when X⁵ represents methyl or methoxy;
(4) X³, X⁵ and X⁷ all represent H, then X⁴ and X⁶ do not both represent —C(O)OCH₃ or —C(O)O-isopropyl;
(5) X⁴, X⁶ and X⁷ all represent H, then X⁵ does not represent F when X³ represents methyl;
(6) X³, X⁶ and X⁷ all represent H, then X⁵ does not represent F when X⁴ represents —NO₂;
(7) X⁴, X⁵ and X⁶ represents H, then X⁷ does not represent isopropyl when X³ represents methyl;
(8) X³, X⁵ and X⁷ represents H, then X⁴ and X⁶ do not both represent methoxy;
(9) X⁴, X⁵, X⁶ and X⁷ all represent H, then X³ does not represent methoxy; or
(B) when R¹ represents H, R² represents CF₃, X⁴, X⁶ and X⁷ all represent H, then X³ does not represent chloro or CF₃ when X⁵ represents —NO₂.

26. A compound or salt as claimed in claim 25, with the additional provisos that, when R² represents CF₃ and:
(I) R¹ represents H or Cl, X⁷ represents H and:
(a) X⁴, X⁵ and X⁶ all represent H, then X³ does not represent CF₃;
(b) X⁴ and X⁶ both represent H, then X³ does not represent bromo when X⁵ represents —NO₂;
(c) X⁴ and X⁵ both represent H, then X³ does not represent chloro when X⁶ represents CF₃;
(d) X⁴ represents H, then X³ does not represent chloro when X⁵ represents —NO₂ and X⁶ represents chloro;
(II) R¹ represents H or Cl, then X³, X⁴, X⁵, X⁶ and X⁷ do not all represent F;
(III) R¹ represents Cl and X⁴, X⁶ and X⁷ all represent H, then X³ does not represent chloro or CF₃ when X⁵ represents —NO₂;
(IV) R¹ represents H, X³ represents Cl, then:
(i) X⁴, X⁵, X⁶ and X⁷ do not all represent H;
(ii) X⁴ does not represent Cl when X⁵ and X⁶ represent H or Cl and X⁷ represents H;
(iii) X⁵ does not represent Cl or Br when X⁴, X⁶ and X⁷ all represent H;
(iv) X⁷ does not represent Cl when X⁵ represents H, Cl or —NO₂ and X⁴ and X⁶ both represent H;
(v) X⁵ does not represent Cl when X⁶ represents Cl and X⁴ and X⁷ both represent H;
(V) R¹ represents H and X³ represents Br, then X⁵ does not represent —OCF₃ when X⁴, X⁶ and X⁷ all represent H;
(VI) R¹ represents H and X³ represents F or 1, then X⁵ does not represent —NO₂ when X⁴, X⁶ and X⁷ all represent H;
(VII) R¹ represents H and X³ represents —NO₂, then X⁵ does not represent C₁ or CF₃ when X⁴, X⁶ and X⁷ all represent H;
(VIII) R¹ represents H, X³ represents CF₃, then X⁵ does not represent —NO₂ when X⁴ and X⁶ both represent H and X⁷ represents Cl; or
(IX) R¹ represents H, X³ represents CF₃, then X⁵ does not represent Cl, when X⁴, X⁶ and X⁷ all represent H.

27. A combination product comprising:
(A) a compound of formula I, as defined in claim 1, or a pharmaceutically-acceptable salt thereof; and
(B) another therapeutic agent that is useful in the treatment of inflammation,
wherein each of components (A) and (B) is formulated in admixture with a pharmaceutically-acceptable adjuvant, diluent or carrier.

28. A combination product as claimed in claim 27 which comprises a pharmaceutical formulation including a compound of formula I as defined in claim 1, or a pharmaceutically-acceptable salt thereof, another therapeutic agent that is useful in the treatment of inflammation, and a pharmaceutically-acceptable adjuvant, diluent or carrier.

29. A combination product as claimed in claim 27 which comprises a kit of parts comprising components:
(a) a pharmaceutical formulation including a compound of formula I as defined in claim 1, or a pharmaceutically-acceptable salt thereof in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

30. A process for the preparation of a compound of formula Ia as defined in claim 25, which comprises:
(i) for compounds of formula I in which R² represents CHF₂, Cl, F or CF₃, reaction of a corresponding compound of formula I in which R² represents hydrogen, with an appropriate base followed by quenching with an appropriate electrophile;
(ii) for compounds of formula I in which R² represents CF₃, reaction of a compound corresponding to a compound of formula I but in which R² represents bromo or iodo with CuCF₃ (or a source of CuCF₃);
(iii) reaction of a compound of formula III, 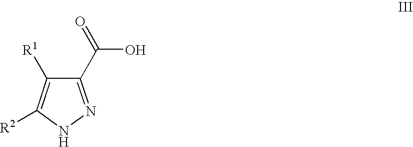
or a N-protected and/or O-protected derivative thereof, wherein R¹ and R² are as defined in claim 1, with a compound of formula IV, 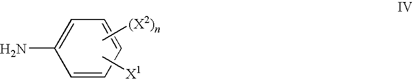
and n are as defined in claim 1;
(iv) reaction of a compound of formula V, 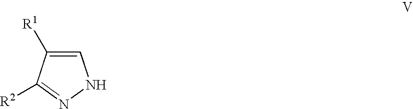
wherein R¹ and R² are as defined in claim 1, with a suitable base followed by reaction with a compound of formula VI, 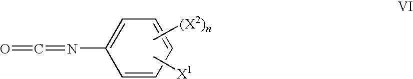
wherein X¹, X² and n are as defined in claim 1, followed by quenching with a suitable proton source;
(v) for compounds of formula I in which R² represents hydrogen and R¹ is as defined in claim 1, removal of the group J from a compound of formula VII, 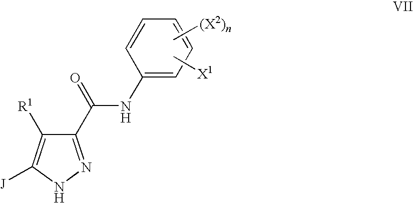
wherein J represents —Si(R^{t})₃ or —Sn(R^{z})₃ (in which each R^{t} independently represents a C₁₋₆ alkyl group or an aryl group and each R^{z} independently represents C₁₋₆ alkyl), and R¹, X¹, X² and n are as defined in claim 1, with an appropriate reagent for the removal of the silyl group (when J represents —Si(R^{t})₃) or by hydrolysis (when J represents —Sn(R^{z})₃);
(vi) reaction of a compound of formula VIII, 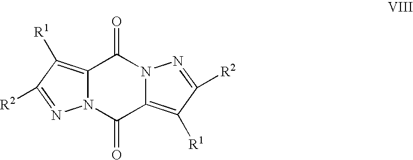
wherein R¹ and R² are as defined in claim 1, with a compound of formula IV as defined above;
(vii) for compounds of formula I in which one of R¹ or R² represents CHF₂, CF₃, Cl or F and the other represents H, reaction of a compound corresponding to a compound of formula I but in which one of R¹ or R² represents bromo or iodo and the other represents H (as appropriate) with a suitable organolithium base optionally in the presence of an additive, followed by quenching with an appropriate electrophile; or
(viii) reaction of a compound of formula VIIIA 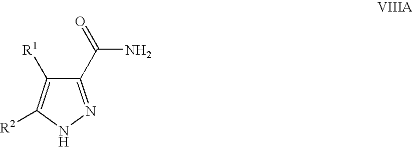
or a N-protected derivative thereof, wherein R¹ and R² are as defined in claim 1, with a compound of formula VIIIB, 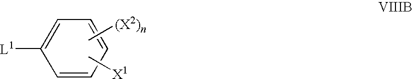
wherein L¹ represents a suitable leaving group and X¹, X² and n are as defined in claim 1.

31. A process for the preparation of a pharmaceutical formulation as defined in claim 19, which process comprises bringing into association a compound of formula I as defined in claim 1, or a pharmaceutically acceptable salt thereof with a pharmaceutically-acceptable adjuvant, diluent or carrier.

32. A process for the preparation of a combination product as defined in claim 27, which process comprises bringing into association a compound of formula I as defined in claim 18, or a pharmaceutically acceptable salt thereof with the other therapeutic agent that is useful in the treatment of inflammation, and at least one pharmaceutically-acceptable adjuvant, diluent or carrier.
